(19) United States
(12) Patent Application Publication
(10) Pub. No.: US 2024/0023590 A1
(43) Pub. Date: Jan. 25, 2024

(54) METHODS FOR INCREASING VISCOSITY OF DIEATRY FIBERS AND SYNERGISTIC COMPOSITIONS THEREFOR

(71) Applicant: perora GmbH, Heidelberg (DE)

(72) Inventor: Dirk Vetter, Heidelberg (DE)

(21) Appl. No.: 18/041,412

(22) PCT Filed: Aug. 13, 2021
(86) PCT No.: PCT/EP2021/072594

§ 371 Date: Feb. 13, 2023

(30) Foreign Application Priority Data
Aug. 14, 2020 (EP) 20191094.0

(51) Int. Cl. A23L 33/125; A23L 33/115

(52) U.S. Cl. CPC A23L 33/125; A23L 33/115

(57) ABSTRACT

The present invention provides a method of modifying the swelling behavior of a dietary fibre in an aqueous medium by providing a synergistic fibre composition comprising the dietary fibre embedded within a lipid material comprising a lipid selected from a free fatty acid exhibiting a melting point in the range of from 30 to 60° C., a glycerol- or sorbitan based fatty acid ester exhibiting a melting point in the range of from 30 to 75° C., a plant-derived wax exhibiting a melting point in the range of from 30 to 75° C., or a homogenous mixture of two or more of these lipids, wherein said homogenous mixture exhibits a melting point in the range of from 30 to 70° C.; wherein the swelling behavior is modified in such a way that the maximum viscosity that is achieved by mixing the synergistic fibre composition, comprising the dietary fibre(s) at an amount x_{_fibre}, in a given volume Vₓ of the aqueous medium (η_{max_syn}) is higher than the maximum viscosity achieved in the same volume Vₓ of an aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}) and higher than the cumulative maximum viscosity of η_{max_fibre} plus η_{max_other} (η_{max_Σ}); and optionally the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h. The present invention further provides respective synergistic fibre compositions exhibiting the modified swelling behavior of the dietary fibre comprised therein; as well as methods of using said synergistic fibre compositions.

### BACKGROUND OF THE INVENTION

Dietary fibres are edible carbohydrate polymers which typically make up the portion of plant-based or plant-derived food that cannot be completely broken down by human digestive enzymes. They are essential in nutrition to support and/or improve digestive processes and in turn general wellbeing and health.

Dietary fibres may be soluble or insoluble in water, each with different physicochemical characteristics and effects on the digestive tract. For instance, most soluble fibres are fermentable by gut bacteria in the colon, while insoluble fibres are inert to digestive enzymes in the upper gastrointestinal tract and only few of them can be fermented. Fermentation can be beneficial in that leads to end products such as short-chain fatty acids which support colonic function and health by promoting bacterial growth and in turn increasing stool volume with the elevated bacterial mass. In addition, both insoluble and soluble fibre may also absorb water, or in other words swell, as they move through the digestive system, thereby also leading to increased stool volumes and improved colon motility.

Certain soluble fibres exhibit a fibre mediated uptake of water that does not only result in a volume increase of the gastrointestinal contents (herein referred to as bulking) but also in a viscosity increase (herein referred to as gelling or thickening). Said fibres are called ‘viscous fibres’ or ‘high-viscosity fibres’, and examples include raw guar gum, beta-glucans, psyllium, pectins, arabinoxylan, galactomannan, glucomannan and modified celluloses like methylcellulose (MC), carboxymethylcellulose (CMC), and hydroxypropylmethyl-cellulose (HPMC). This increased viscosity of the gastrointestinal contents, and in particular of the chyme, delays gastric emptying which can result in an extended feeling of fullness in humans. It also reduces the contact of digestive enzymes with food components, leading to slower break down and delayed resorption of nutrients.

This fibre-mediated increased viscosity of the gastrointestinal contents (e.g. the chyme or stool) is used in a variety of therapeutic settings. In the treatment of patients with elevated plasma cholesterol levels, for instance, a higher viscosity of the chyme has been shown to lead to decreased reuptake of bile acids and cholesterol, resulting in a loss of bile acids that is then compensated through de-novo bile acid synthesis from cholesterol, thereby leading to lower blood LDL-cholesterol levels. Viscous fibres are also beneficially used with diabetes patients, especially for type 2 diabetes, where the viscosity-mediated delay of glucose uptake results in decreased postprandial blood glucose levels (e.g. a decreased maximum plasma concentration of glucose after meals), decreased HbA1c-levels as well as lower inflammation parameters. The use of viscouse fibres thus diminishes deleterious hyperglycemia and its consequences. Furthermore, viscous fibres can improve insulin-sensitivity and contribute to a reduction in the dosage of oral anti-diabetic medication.

The effect of viscosity-induced slower break down and delayed resorption of nutrients is more pronounced if the viscous fibre has a higher non-fermentable proportion such as seen, for example, with psyllium products (i.e. plantago seed husks), carboxymethylcellulose (CMC) and hydroxypropylmethylcellulose (HPMC). Furthermore, it is known that the extent of the positive effects and health benefits of these fibres correlate with the magnitude of viscosity; for instance, with higher viscosities being more potent to delay nutrient absorption. Viscosity is therefore a key parameter for dietary fibres' efficacy and/or fibre-mediated health benefits.

A major drawback, tough, is that, in general, viscous fibres are not easy to ingest in effective quantities; for instance, depending on the particular fibre, in order to yield a sufficient ‘thickening effect’ of the chyme in the digestive tract there have to be amounts consumed between 2 g and 15 g of soluble fibre per dosage up to 3 times daily. Furthermore, upon preparation and/or ingestion, viscous fibres may form lumps, or a pudding-like viscosity too thick to drink; or they may yield a somewhat ‘slimy’ mouthfeel. In consequence, viscous fibre products may lead to low compliance with respect to regular intake and adequate dosage.

Several efforts have been made to accomplish more palatable compositions of viscous fibres which overcome the above disadvantages. Some authors describe the use of high amounts of sugar or maltodextrin to retard the gelling and facilitate dispersion. This is not desirable, of course, in view of the negative health effects of daily sugar, especially for people suffering from, or being at risk for, diabetes, in particular diabetes type 2. Other approaches are focused on specific particle sizes of the viscous fibres (e.g. cellulose ethers), or on dry powder mixes for optimized dispersion- and dissolution properties.

There is still a need for improved compositions for the administration of soluble dietary fibres, in particular the oral administration of viscous fibres, which allow for both easy dispersion and a palatable, pleasant consistency upon ingestion. It is thus an object of the present invention to provide said compositions. It is also an object of the present invention to provide means, or compositions, to increase the maximum viscosity obtainable with a given type and amount of viscous fibre so as to increase its effectiveness and potentially lower the dosage necessary to yield the desired effects. Further objects of the invention will be clear on the basis of the following description of the invention, the examples, and claims.

### SUMMARY OF THE INVENTION

In a first aspect, the present invention provides a synergistic fibre composition comprising (a) a dietary fibre selected from the group consisting of hydroxypropylmethylcellulose (HPMC), methylcellulose (MC), carboxymethyl cellulose (CMC), psyllium seed husk, xanthan gum, galactomannan, and glucomannan; and (b) a lipid material comprising a lipid selected from (b₁) a free fatty acid exhibiting a melting point in the range of from 30 to 60° C., (b₂) a glycerol- or sorbitan based fatty acid ester exhibiting a melting point in the range of from 30 to 75° C., (b₃) a plant-derived wax exhibiting a melting point in the range of from 30 to 75° C., or a homogenous mixture of two or more of the lipids under b₁, b₂ and/or b₃, wherein the homogenous mixture of these lipids exhibits a melting point in the range of from 30 to 70° C., wherein the lipid material under (b) comprises at least one lipid under b₁ to b₃ which is selected from the group consisting of myristic acid, capric acid, glycerolmonostearate (GMS), tristearin, and candelilla wax; wherein the dietary fibre is embedded within the lipid material; and wherein the synergistic fibre composition is capable of modifying the swelling behavior of the dietary fibre in such a way that:
the maximum viscosity that is achieved by mixing the synergistic fibre composition, comprising the dietary fibre(s) at an amount x_{_fibre}, in a given volume Vₓ of an aqueous medium (η_{max_syn}) is higher than the maximum viscosity achieved in the same volume Vₓ of the aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}), and higher than the cumulative maximum viscosity of η_{max_fibre} plus η_{max_other} (η_{max_Σ}); and optionally
the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.

In a second aspect, the invention provides a method of modifying the swelling behavior of a dietary fibre in an aqueous medium by providing a synergistic fibre composition comprising (a) the dietary fibre, and (b) a lipid material comprising a lipid selected from (b₁) a free fatty acid exhibiting a melting point in the range of from 30 to 60° C., (b₂) a glycerol- or sorbitan based fatty acid ester exhibiting a melting point in the range of from 30 to 75° C., (b₃) a plant-derived wax exhibiting a melting point in the range of from 30 to 75° C., or a homogenous mixture of two or more of the lipids under b₁, b₂ and/or b₃, wherein the homogenous mixture of these lipids exhibits a melting point in the range of from 30 to 70° C.; wherein the dietary fibre is embedded within the lipid material, and wherein the swelling behavior is modified in such a way that:
the maximum viscosity that is achieved by mixing the synergistic fibre composition, comprising the dietary fibre(s) at an amount x_{_fibre}, in a given volume Vₓ of the aqueous medium (η_{max_syn}) is higher than the maximum viscosity achieved in the same volume Vₓ of the aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}), and higher than the cumulative maximum viscosity of η_{max_fibre} plus η_{max_other} (η_{max_Σ}); and optionally
the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.

In a third aspect, the invention relates to a process for preparing the synergistic fibre composition provided for the method according to the second aspect of the invention, or specifically the synergistic fibre composition according to the first aspect of the invention, wherein the process comprises a step of processing a blend comprising the dietary fibre under (a) and the lipid material under (b) by:
(i) extruding, or melt-extruding, the blend;
(ii) spray-congealing the blend, optionally using a jet-break-up technique;
(iii) melt-granulating the blend;
(iv) compressing the blend into minitablets;
(v) melt-injecting the blend into a liquid medium; or
(vi) spray-coating the blend onto inert cores.

In a fourth aspect, the invention relates to the synergistic fibre composition provided for the method according to the second aspect of the invention, or specifically the synergistic fibre composition according to the first aspect of the invention, for use as a medicament.

In a fifth aspect, the invention relates to the synergistic fibre composition provided for the method according to the second aspect of the invention, or specifically the synergistic fibre composition according to the first aspect of the invention, for use in the treatment and/or prevention of gastro-intestinal and/or metabolic disorders.

In a sixth aspect of the invention, the invention relates to a solid dietary fibre formulation for oral administration comprising, or consisting of, the synergistic fibre composition provided for the method according to the second aspect of the invention, or specifically the synergistic fibre composition according to the first aspect of the invention, wherein the synergistic fibre composition is provided in the form of a plurality of dry, flowable, ingestible particles, for instance as minitablets, granules, pellets, or powders, or has been prepared from said plurality of dry, flowable, ingestible particles.

### BRIEF DESCRIPTION OF THE FIGURES

FIG. 1 depicts the effect of embedding the dietary fibre into lipids according to the invention, with FIG. 1a showing characteristic viscosity kinetics of pure HPMC powder (Nr. 1) and HPMC melt-granulated at lab-scale with a 2:1 GMS/GMO mixture (Nr. 5), and FIG. 1b showing characteristic viscosity kinetics of pure KGLM powder (Nr. 34) and KGLM melt-granulated at lab-scale with a 2:1 GMS/GMO mixture (Nr. 36);

FIG. 2 shows the exemplary viscosity kinetics of test Nr. 2 (HPMC melt-granulated at lab-scale with GMS) and Nr. 72 (HPMC melt-extruded at lab-scale with capric acid);

FIG. 3 depicts the effect of blending a higher-melting lipid such as GMS (Mp 71-72° C.) with a lower-melting lipid such as GMO (Mp 30° C.) by showing the exemplary viscosity kinetics of test Nr. 2 (HPMC melt-granulated at lab-scale with GMS) and Nr. 5 (HPMC melt-granulated at lab-scale with GMS/GMO);

FIG. 4 depicts the effects of i) extrusion and ii) lecithin addition to the lipids by showing the exemplary viscosity kinetics of test Nr. 5 (HPMC melt-granulated at lab-scale with GMS/GMO) and Nr. 8 (HPMC melt-extruded at lab-scale with GMS/GMO), as well the respective tests Nr. 6 and 9 with added lecithin;

FIG. 5 depicts the effects of xanthan additions; with FIG. 5a showing the effects of xanthan additions to a melt-granulated core and/or to its surface (in form of a powder layer top coating) by showing the exemplary viscosity kinetics of test Nr. 5 (HPMC/GMS/GMO 40/40/20), test Nr. 43 (HPMC/X/GMS/GMO 39/2/39/20; i.e. same as Nr. 5 but with an addition of xanthan to the core), as well as tests Nr. 45 and 44 (same as Nr. 5 and Nr. 43, respectively, but exhibiting a 4% xanthan powder-layer top coating); and with FIG. 5b showing the effects of xanthan additions to a melt-extruded core and/or to its surface (in form of a powder layer top coating) by showing the exemplary viscosity kinetics of test Nr. 64 (HPMC/X/GMS/liquid GMO 39/2/54/5+2% xanthan powder-layer) and test Nr. 91 (HPMC/GMS/liquid GMO 40/55/5+4% xanthan powder-layer).

### DEFINITIONS

The following terms or expressions as used herein should normally be interpreted as outlined in this section, unless explicitly defined otherwise by the description or unless the specific context clearly indicates or requires otherwise.

All technical terms as used herein shall be understood to have the same meaning as is commonly understood by a person skilled in the relevant technical field.

The words ‘comprise’, ‘comprises’ and ‘comprising’ and similar expressions are to be construed in an open and inclusive sense, as ‘including, but not limited to’ in this description and in the claims.

The singular forms ‘a’, ‘an’ and ‘the’ should be understood as to include plural referents. In other words, all references to singular characteristics or limitations of the present disclosure shall include the corresponding plural characteristic or limitation, and vice versa. The terms ‘a’‘an’ and ‘the’ hence have the same meaning as ‘at least one’ or as ‘one or more’. For example, reference to ‘an ingredient’ includes mixtures of ingredients, and the like.

The expressions, ‘an embodiment’, ‘one embodiment’, or ‘specific embodiments’ and the like mean that a particular feature, property or characteristic, or a particular group, or combination, of features, properties or characteristics, as referred to in combination with the respective expression, is present in at least one of the embodiments of the invention. These expressions, occurring in various places throughout this description, do not necessarily refer to the same embodiment. Moreover, the particular features, properties or characteristics may be combined in any suitable manner in one or more embodiments.

The terms ‘consisting of’, ‘substantially consisting of’ or ‘essentially consisting of’ mean that no further components are added to a composition or dosage form other than those listed. Nevertheless, very small amounts of other materials may potentially be present, such as material-inherent impurities. Furthermore, when referring to e.g. a composition ‘essentially consisting of A, B, and optionally C’, this means that no further components are added to a composition other than A, B and C, yet with C being an optional component (i.e. not mandatory) in said composition.

All percentages, parts and/or ratios in the context of numbers should be understood as relative to the total number of the respective items. Furthermore, all percentages parts and/or ratios are intended to be by weight of the total weight; i.e. ‘%’ should be read as ‘wt.-%’.

The term ‘substantially free of X’ means that the respective material (e.g. a chemical compound or a composition) contains less than a functional amount of the optional ingredient X, typically less than 5 wt.-%, or less than 1 wt.-%, preferably less than 0.1 wt.-% or even less than 0.01 wt.-%, and also including 0 wt.-% of the respective ingredient X. The expression refers, inter alia, to very small amounts of the respective ingredient X, such as material-inherent impurities or residual moisture, which may potentially be present in (raw) materials despite the aim to render a material completely free of them. For example, ‘substantially free of water’ means that no water is deliberately included in a material but does not exclude the presence of residual moisture.

Terms such as ‘about’, ‘approximately’, ‘ca.’, ‘essentially’, ‘substantially’ are meant to compensate for the variability allowed for in the pharmaceutical industry and inherent in pharmaceutical products, such as differences in content due to manufacturing variation and/or time-induced product degradation. The terms in connection with an attribute or value include the exact attribute or the precise value, as well as any attribute or value typically considered to fall within a normal range or variability accepted in the technical field concerned.

The term ‘room temperature’ shall be understood as ranging from 15° C. to 25° C. (i.e. 20±5° C.), as is for instance defined by the European Pharmacopoeia or by the WHO guidance ‘Guidelines for the Storage of Essential Medicines and Other Health Commodities” (2003). Other temperature provisions, such as ‘about 37° C.’, and any other temperature provisions implying a controlled environment (such as in an oven, or in a standardized release tester like a USP Dissolution Apparatus type 2 paddle apparatus), are usually understood to be narrower; for instance, depending on the technical parameters of the device used ‘about 37° C.’ means 37±1° C., or even 37±0.5° C.

The terms ‘embedded’ or ‘embedded within’ mean that the dietary fibre is largely dispersed within the lipid material, whether molecularly, colloidally or in the form of a solid suspension. The lipid material forms a continuous, coherent phase in which the dietary fibre is discontinuous and in dispersed form, i.e. a so-called matrix composition.

And while a small fraction of the embedded dietary fibre may show at the outer surface of the synergistic fibre composition (depending, for instance, on factors such as the weight ratio of dietary fibre to lipid material, the processing method, and/or the surface-to-volume ratio of the particle(s)), said surface is predominantly lipidic in nature and formed by the lipid material. In cases where this predominantly lipidic surface is covered by another material layer, such top-coating will be explicitly mentioned. So-called reservoir compositions, however, i.e. compositions exhibiting an inner dietary fibre core covered by an outer lipid coating, are not meant to fall under the definition of ‘a dietary fibre embedded within a lipid material’ as used herein.

Typically, ‘embedded’ in the context of the invention also means that the lipid material and the dietary fibre are mixed so intimately that the porosity of the resulting synergistic lipid-fibre compositions is greatly reduced as compared to compositions formed from the dietary fibre, for instance, by dry granulation (e.g. roller compaction) or wet granulation (e.g. agglomeration with an aqueous or hydroalcoholic binder solution). Particle porosity may be determined by porosimetry, an analytical technique used to determine various quantifiable aspects of a material's porous nature, such as pore diameter, total pore volume, and surface area. The technique involves the intrusion of a non-wetting liquid at high pressure into a material through the use of a porosimeter.

The term ‘lag time’ refers to the time period at the onset of swelling in which the viscosity does not yet exceed 5% of the maximum viscosity. This lag time is desirable to avoid excessive and/or rapidly forming viscosity during the oral administration step.

When referring to ‘dietary fibre(s) alone’ (such as in “the maximum viscosity achieved in the same volume Vx of the aqueous medium by the dietary fibre(s) alone”), this expression refers to the dietary fibre(s) when employed without being formulated with the lipid material(s) under (b); for instance, the dietary fibre(s) without being embedded within said lipid material(s). This can be, for instance, a dry powder of hydroxypropylmethylcellulose (HPMC), or a dry powder mix of HPMC and xanthan.

The term ‘melting point’ refers to the lipid material(s) under (b) as such, i.e. not in its hydrated state, and it should be understood as the temperature at which the lipid material melts entirely, without solid residue, at normal pressure. In case, a lipid material exhibits a broad melting range, the melting point is understood as the higher limit of the range.

The term ‘sieve diameter’ of a particle, e.g. a sieve diameter in the range of 0.01 mm to 3 mm, means that the particle would normally pass through a sieve having an aperture or opening size of about 3 mm, but not through a sieve having an aperture or opening size of about 0.01 mm or less. In case of a plurality of particles, particle size provisions in terms of sieve diameters should be interpreted to characterise the mass median sieve diameter of said plurality of particles; or, in other words, at least the mass median sieve diameter of a plurality of particles should comply with a given particle size provision, e.g. falling within the range of 0.01 mm-3.0 mm.

The term ‘swelling’, such as in ‘swelling behaviour’, refers refers to the uptake of water or an aqueous medium by the initially solid dietary fibre in an aqueous environment, caused by an influx, or diffusion process, of water accompanied by hydration, and typically rendering said medium viscous (i.e. increasing viscosity) and leading to a volume increase of the solid fibre (‘bulking’). Swelling may be expressed, for instance, by the swelling value in percent calculated as (wₛ−w_{d})/w_{d}×100 (w_{d}=initial weight of the dry component and wₛ=weight of swollen component). Optionally, swelling may be evaluated under conditions mimicking in vivo conditions; e.g. by testing swelling behaviour in simulated gastric or intestinal fluids, such as fasted-state simulated intestinal fluid (FaSSGF).

The term ‘flowable’ refers to compositions, or formulations comprising these compositions, which are present in a physical form that allows for their gravity-driven and efficient transfer by pouring from, or emptying of, a container housing them; e.g. after opening the container and tilting it or turning it upside down. Typically, compositions, or formulations comprising them, which come in the form of solid particles exhibiting a sieve diameter in the range of 0.01 mm-3.0 mm are considered flowable when composed as described herein.

The term ‘synthetic drug substance’ refers to substances, namely active pharmaceutical ingredient (APIs) which are administered, e.g. ingested, so as to cause an intentional therapeutic effect (i.e. an intentional change in a subject's physiology or, in some cases, psychology), and which are produced or obtained by chemical synthesis. In other words, ‘synthetic drug substances’, as understood herein, typically do not occur naturally. Furthermore, synthetic drug substances are commonly understood as being distinguished from, for instance, food, dietary supplements, and other substances that provide nutritional support; such as dietary fibres of the present invention.

The terms ‘viscous fibre’ or ‘viscous dietary fibre’ refer to dietary fibres according to the present invention and to fibres which yield a viscosity of more than 10 mPas, preferably more than 100 mPas, in water at room temperature when prepared as a 5 wt.-% solution, and more preferably when prepared as a 2 wt.-% solution.

The term ‘guar’, or ‘guar gum’, refers to raw guar, or, in other words, to non-hydrolyzed guar; for instance, a guar that yields a viscosity of more than 10 mPas, preferably more than 100 mPas, in water at room temperature when prepared as a 5 wt.-% solution. Guar gum is one of the suitable sources for galactomannans, an exemplary viscous dietary fibre according to the invention. The term ‘galactomannans’ refers to viscous poly-saccharides (gums) consisting of polymannose substituted with galactose which are found in the endosperm of plant seeds, such as fenugreek gum, guar gum, tara gum, locust bean gum or carob gum, or cassia gum.

The term ‘glucomannans’ refers to polysaccacharides consisting of polymerized β-(1→4) linked mannose and glucose units found for instance in the roots of the konjac plant or tubers of certain orchid species (e.g. salep flour).

### DETAILED DESCRIPTION OF THE INVENTION

In a first aspect, the present invention provides a synergistic fibre composition comprising (a) a dietary fibre selected from the group consisting of hydroxypropyl-methylcellulose (HPMC), methylcellulose (MC), carboxymethylcellulose (CMC), psyllium seed husk, xanthan gum, galactomannan (e.g. from guar gum or locust bean gum), and glucomannan (e.g. from konjac gum); and (b) a lipid material comprising a lipid selected from (b₁) a free fatty acid exhibiting a melting point in the range of from 30 to 60° C., (b₂) a glycerol- or sorbitan based fatty acid ester exhibiting a melting point in the range of from 30 to 75° C., (b₃) a plant-derived wax exhibiting a melting point in the range of from 30 to 75° C., or a homogenous mixture of two or more of the lipids under b₁, b₂ and/or b₃, wherein the homogenous mixture of these lipids exhibits a melting point in the range of from 30 to 70° C., wherein the lipid material under (b) comprises at least one lipid under b₁ to b₃ which is selected from the group consisting of myristic acid, capric acid, glycerolmonostearate (GMS), tristearin, and candelilla wax; wherein the dietary fibre is embedded within the lipid material; and wherein the synergistic fibre composition is capable of modifying the swelling behavior of the dietary fibre in such a way that:
the maximum viscosity that is achieved by mixing the synergistic fibre composition, comprising the dietary fibre(s) at an amount x_{_fibre}, in a given volume Vₓ of an aqueous medium (η_{max_syn}) is higher than the maximum viscosity achieved in the same volume Vₓ of the aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}) and higher than the cumulative maximum viscosity of η_{max_fibre} plus η_{max_other} (η_{max_Σ}); and optionally
the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.

In other words, the present invention relates to synergistic fibre compositions of soluble dietary fibres, in particular so-called viscous fibres, or high-viscosity fibres, which are formulated with further components, such as non-viscosity-increasing lipids, in such a way that the composition, when dispersed, and ultimately dissolved, in water or aqueous solutions, exhibits (i) a maximum viscosity higher than the viscosities achieved by its singular components and the sum thereof; and optionally (ii) sigmoidal viscosity kinetics. The present invention thus differs from previous approaches in that it facilitates dispersion of the fibre composition, and provides a palatable, pleasant consistency upon its dispersion in water, or other ingestible, aqueous media, by formulating viscous fibres with lipids, and, more specifically, by embedding the viscous fibres within the lipid(s).

For reasons of brevity, the following variables will be used throughout this application:
x_{_fibre} a given amount x of the dietary fibre(s), e.g. in g or mg
x_{_syn} the corresponding amount x of the synergistic fibre composition comprising the dietary fibre(s) at amount x_{_fibre}, e.g. in g or mg
x_{_other}/x_{_lipid} the corresponding amount x of other component(s) (e.g. of the lipid material under (b)) comprised in amount x_{_syn}, e.g. in g or mg (where there are no components besides the dietary fibre(s) other than the lipids, then x_{_other} can sometimes also be referred to as x_{_lipid})
Vₓ a given volume V of the aqueous medium, e.g. in mL or L, in which said amount x_{_fibre} is mixed (either the dietary fibre alone, or the dietary fibre comprised in the synergistic fibre composition)
ηₘₐₓ a maximum viscosity, e.g. in mPas
η_{max_syn} the maximum viscosity achieved by mixing the synergistic fibre composition comprising the dietary fibre(s) at amount x_{_fibre} in volume Vₓ
η_{max_fibre} the maximum viscosity achieved by mixing the dietary fibre(s) alone at amount x_{_fibre} in volume Vₓ
η_{max_other} the maximum viscosity achieved by mixing any of the other components of the synergistic fibre composition (in particular, by the lipid material under (b)) at amounts x_{_other}, or as the case may be x_{_lipid}, in volume Vₓ η_{max_Σ} the cumulative maximum viscosity of η_{max_fibre} plus η_{max_other}; or, in other words, the sum of the maximum viscosities of all singular components of the synergistic fibre composition (as achieved by mixing the corresponding amount(s) x of said components as comprised in amount x_{_syn} in volume Vₓ)
t_{1/2} ½ of the time until reaching ηₘₐₓ (e.g. η_{max_syn} or η_{max_fibre})
t_{1/4} ¼ of the time until reaching ηₘₐₓ (e.g. η_{max_syn} or η_{max_fibre})

This means that, for instance, in case of an illustrative example of a synergistic fibre composition comprising an amount x_{_fibre} of 4 g hydroxypropylmethylcellulose (HPMC) as the dietary fibre embedded within an amount x_{_lipid} of 6 g glycerol monostearate (GMS) as the lipid material, a total amount x_{_syn} of 10 g of said synergistic fibre composition mixed into e.g. 600 mL water (Vₓ) results in a higher maximum viscosity (η_{max_syn}) than (i) the maximum viscosity achieved when mixing 4 g HPMC alone in the same volume Vₓ of 600 mL water (η_{max_fibre}), (ii) higher than the maximum viscosity achieved when mixing 6 g GMS in a Vₓ of 600 mL water (η_{max_lipid}), and also (iii) higher than the cumulative maximum viscosity of η_{max_fibre} plus η_{max_other} (η_{max_Σ}).

Alternatively, in case of an illustrative example of a synergistic fibre composition comprising an amount x_{_fibre} of 4 g HPMC plus 0.4 g xanthan as the dietary fibres embedded within an amount x_{_lipid} of 4 g GMS plus 2 g glycerol monooleate (GMO) as the lipid materials, then a total amount x_{_syn} of 10.4 g of said synergistic fibre composition mixed into e.g. 600 mL water (Vₓ) results in a higher maximum viscosity (η_{max_syn}) than (i) the maximum viscosity achieved when mixing the two dietary fibres (4 g HPMC+0.4 g xanthan) alone in the same volume Vₓ of 600 mL water (η_{max_fibre}), (ii) higher than the maximum viscosity achieved when mixing 4 g GMS and 2 g GMO in a Vₓ of 600 mL water (η_{max_lipid}), and also (iii) higher than the cumulative maximum viscosity of η_{max_fibre} plus η_{max_other} (η_{max_Σ}).

This finding of the present invention is surprising in so far that lipids, such as the lipids under b₁ to b₃, or in the above examples the GMS or GMS/GMO, are typically inert and immisible with water, and are, thus, not commonly known as materials that are capable of increasing the maximum viscosity achievable in aqueous media with dietary fibres; or as materials that are capable of introducing increased viscosity in aqueous media themselves (at least not to a clinically relevant degree).

Without wishing to be bound by theory, it is believed that the underlying mechanism of these synergistic increases of the maximum viscosity may involve the homogenous distribution of the dietary fibres, e.g. HPMC, in the lipids of the synergistic fibre compositions which promotes a far more homogenous dispersion of the viscous fibre in aqueous media such as water, and may thus facilitate a constant and fast hydration, and ultimately dissolution, of said fibre. In other words, it is assumed that it is due to formulating the dietary fibre(s) in the lipid(s) under (b), more specifically by embedding them therein, that the synergistic fibre composition is rendered capable of modifying the dietary fibre(s)' swelling behavior as claimed.

In one of the preferred embodiments, the synergistic fibre composition according to this first aspect of the invention, exhibits both viscosity modifying features; i.e. it is capable of modifying the swelling behavior of the dietary fibre in such a way that:
η_{max_syn} is higher than the η_{max_fibre}, higher than η_{max_other}, and higher than η_{max_Σ}; and
the swelling exhibits sigmoidal viscosity kinetics characterized by a lag time of at least 15 min, followed by a viscosity increase to at least 80% of η_{max_syn} within no more than 2 h.

A synergistic fibre composition providing both these swelling parameters (i.e. increased maximum viscosity and sigmoidal viscosity kinetics) is advantageous in so far as the increased viscosity maximum achievable with the composition does not negatively impact the dispersion behaviour of the composition or its mouthfeel or ease of ingestion; for instance, the synergistic fibre composition does not form more lumps, or feels even thicker upon ingestion, than the prior art compositions of the same fibre. Instead, the sigmoidal viscosity increase provides a lag phase that allows for sufficient time to prepare and ingest the synergistic fibre composition, dispersed in water, or another ingestible, aqueous medium, without premature gelling, while also allowing for an effective viscosity increase after the fibre has reached the stomach and/or the upper intestine

In specific embodiments, the weight ratio of the dietary fibre under (a) to the lipid material under (b) in the synergistic fibre composition is in the range from 30:70 to 60:40, or from 33:67 to 55:45, or from 35:65 to 50:50, or from 35:65 to 45:55, or from 37:63 to 42:58; preferably from 35:65 to 50:50.

In further specific embodiments, the synergistic fibre composition comprises at least 45 wt.-%, or at least 50 wt.-%, or at least 55 wt.-%, or at least 60 wt.-% of the lipid material under (b) based on the total weight of the synergistic fibre composition.

In yet further specific embodiments, the synergistic fibre composition comprises at least 50 wt.-% of the lipid material under (b) based on the total weight of the synergistic fibre composition; and the weight ratio of the dietary fibre under (a) to the lipid material under (b) in the synergistic fibre composition is in the range from 35:65 to 50:50.

The synergistic fibre composition according to the first aspect of the invention may be prepared by processing a blend comprising the dietary fibre under (a) and the lipid material under (b) in such a way that the dietary fibre is embedded within the lipid materials, preferably, in such a way that the resulting processed blend exhibits a greatly reduced porosity compared to, for instance, compositions obtained from mere dry-granulation of the dietary fibre, or wet-granulation thereof with an aqueous or hydroalcoholic binder solution. The may, for instance, be prepared by (i) extruding, or melt-extruding, the blend (e.g. using a screw extruder); (ii) spray-congealing the blend, optionally using a jet-break-up technique; (iii) melt-granulating the blend; (iv) compressing the blend into minitablets; (v) melt-injecting the blend into a liquid medium; or (vi) spray-coating the blend onto inert cores. In one of the preferred embodiments of the synergistic fibre composition, the dietary fibre under (a) is melt-embedded within, optionally melt-extruded with, the lipid material under (b).

In specific embodiments, the synergistic fibre composition does not comprise any dietary fibres other than a dietary fibre selected from the group consisting of hydroxypropylmethylcellulose (HPMC), methylcellulose (MC), carboxymethylcellulose (CMC), psyllium seed husk, xanthan gum, galactomannan, and glucomannan.

In further specific embodiments, the synergistic fibre composition is substantially free of poly(carboxylates), poly(methacrylic acid), copolymers of acrylic and methacrylic acid, poly(hydroxyethyl methacrylic acid), alginic acid or salts thereof, microcrystalline cellulose, chitosan, gum arabic, beta glucan and pectins.

In specific embodiments, the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, of a lipid selected from the group consisting of monoglycerides, triglycerides, and sorbitan-fatty acid triesters, based on the total weight of the lipid material (b).

In further specific embodiments, the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b).

In specific embodiments, the lipid material under (b) further comprises an additional lipid (b′) in a homogenous mixture with the one or more lipid(s) under b₁, b₂ and/or b₃, wherein additional lipid (b′) is selected from the group consisting of glycerol, a phosphatidylcholine, a sorbitan mono fatty acid ester, one or more fatty acid ester(s) selected from the group consisting of fatty acid ester sof acetic acid, lactic acid, citric acid, tartaric acid, monoacetyl tartaric acid and diacetyl tartaric acid, one or more fatty acids with a melting point below 30° C.; and the homogenous mixture exhibits a melting point in the range of from 30 to 70° C. In more specific embodiments, the additional lipid (b′) comprised in the lipid material under (b) in a homogenous mixture with the one or more lipid(s) under b₁, b₂ and/or b₃, is a phosphatidylcholine, optionally a lecithin such as egg lecithin, soy lecithin, or sunflower lecithin. Alternatively, or in addition to the phosphatidylcholine, the additional lipid (b′) comprised in the lipid material under (b) in a homogenous mixture with the one or more lipid(s) under b₁, b₂ and/or b₃, is a sorbitan mono fatty acid ester, optionally a sorbitan mono fatty acid ester selected from the group consisting of sorbitanmonooleate, polyoxyethylen sorbitanmonolaurate, polyoxyethylen sorbitanmonostearate, and polyoxyethylen sorbitanmonooleate.

In specific embodiments, the lipid material under (b₁) is selected from the group consisting of saturated fatty acids, C₈-C₁₄ fatty acids, and saturated C₈-C₁₄ fatty acids. In more specific embodiments, the lipid material under (b₁) is selected from the group consisting of myristic acid and capric acid.

In specific embodiments, the lipid material under (b₃) is a plant-derived wax selected from the group consisting of candelilla wax, carnauba wax, berry wax, myrica fruit wax, soy wax, rice bran wax, and bees wax.

In specific embodiments, the lipid material under (b) comprises at least 70 wt.-%, or at least 75 wt.-%, or at least 80 wt.-%, or at least 85 wt.-%, or at least 90 wt.-%, of the lipids (b₁) to (b₃) based on the total weight of the lipid material (b). In more specific embodiments, the lipid material under (b) consists of the lipids (b₁) to (b₃), or optionally of the lipids (b₁) to (b₃) and the additional lipids (b′).

In one of the preferred embodiments of the synergistic fibre composition according to the first aspect of the invention, the lipid material under (b) exhibits a melting point in the range of from 30 to 70° C.

In specific embodiments, the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b); and the lipid material under (b) comprises at least one other lipid selected from the group consisting of caprylic acid, glycerol, and glycerolmonooleate (GMO).

It was observed that in some cases, the addition of lower-melting lipids (such as GMO/Mp 30° C., glycerol/Mp 18° C., caprylic acid/Mp 16° C.) to higher-melting lipids (such as myristic acid/Mp˜53° C., GMS/Mp˜71-72° C., tristearin/72-73° C., or candelilla wax/Mp 69-70° C.) yielded beneficial results in that it led to a further increase in the maximum viscosity, and often to shorter t_{1/2} and t_{1/4} values, compared to synergistic fibre compositions using the higher-melting lipids alone. The shorter t_{1/2} and t_{1/4} times by addition of lower-melting lipids can be a valuable tool in cases where the swelling of a composition occurs slower than intended (e.g. if the lag time exceeds 1 h, and/or if after 2 h only about 20% of the maximum viscosity have been reached).

In more specific embodiments, the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmono-stearate (GMS), tristearin, and candelilla wax, based on the total weight of the lipid material (b); and the lipid material under (b) comprises at least one other lipid selected from the group consisting of caprylic acid, glycerol, and glycerolmonooleate (GMO); and the lipid material under (b) further comprises lecithin.

It was observed that in some cases, the addition of small amounts of lecithin, such as 1-5 wt.-%, or 2-4 wt.-%, lecithin based on the amount of the lipid(s), yielded beneficial results in that it led to a further increase in the maximum viscosity compared to the synergistic fibre compositions without the lecithin addition. Furthermore, it was possible to prolong the t_{1/2} and t_{1/4} values by the addition of lecithin, which renders it a valuable tool in cases where the swelling of a composition occurs faster than intended (e.g. with a lag time shorter than 15 min).

In specific embodiments, the synergistic fibre composition according to the first aspect of the invention further comprises xanthan, either in the form of a powder top-coating layer, and/or with the xanthan being embedded in the lipid(s) under (b) together with the dietary fibre under (a). In a more specific embodiment, the synergistic fibre composition further comprises xanthan and the further dietary fibre under (a) in a weight ratio of fibre to xanthan in the range of 98:2 to 75:25, 96:4 to 80:20, or 94:6 to 82:18; such as 95:5, 91:9 or 85:15. In a yet more specific embodiment, the synergistic fibre composition further comprises xanthan in the form of a powder top-coating layer, and the coating level thereof is in the range of 1 to 20 wt.-%, or 1 to 10 wt.-%, or 2 to 8 wt.-%, based on the total weight of the uncoated cores.

In one of the preferred embodiments of the synergistic fibre composition according to the first aspect of the invention, the synergistic fibre composition comprises, or consists of, hydroxpropylmethylcellulose (HPMC) as the dietary fibre under (a); and glycerolmonostearate (GMS), glycerolmonooleate (GMO), and lecithin as the components of the lipid material under (b). In more specific embodiments, the synergistic fibre composition comprises, or consists of, hydroxypropylmethylcellulose (HPMC) and xanthan as the dietary fibres under (a); and glycerolmonostearate (GMS), glycerolmonooleate (GMO), and lecithin as the components of the lipid material under (b). In yet more specific embodiments, the synergistic fibre composition comprises, or consists of, hydroxypropylmethylcellulose (HPMC) and xanthan as the dietary fibres under (a); glycerolmonostearate (GMS), glycerolmonooleate (GMO), and lecithin as the components of the lipid material under (b); and the xanthan is provided either in the form of a powder top-coating layer on the synergistic fibre composition, and/or embedded within the lipid material together with the hydroxypropyl-methylcellulose (HPMC). In yet more specific embodiments, the synergistic fibre composition comprises, or consists of, hydroxypropylmethylcellulose (HPMC) and xanthan as the dietary fibres under (a); glycerolmonostearate (GMS), glycerolmonooleate (GMO), and lecithin as the components of the lipid material under (b); and the xanthan is provided in the form of a powder top-coating layer on the synergistic fibre composition.

Synergistic fibre compositions comprising both GMS and xanthan, or in the above embodiment specifically GMS/GMO and xanthan, are considered favourable in that they provide good wettability of the respective composition, and in particular of the lipid(s) under (b), which form most of the composition's outer surface. A surface that is easily hydrated is preferable in that it facilitates the homogenous dispersion of the synergistic fibre compositions, and ultimately the dietary fibre comprised therein, in aqueous media.

For instance, in one of the preferred embodiments, the synergistic fibre composition, based on its total weight, comprises, or consists of, from 25 to 55 wt.-%, or from 35 to 45 wt.-%, hydroxypropylmethylcellulose (HPMC), from 25 to 55 wt.-%, or from 35 to 45 wt.-%, glycerolmonostearate (GMS), from 10 to 30 wt.-%, or from 15 to 25 wt.-%, glycerolmonooleate (GMO), from 1 to 20 wt.-%, or from 1 to 10 wt.-%, lecithin, and from 1 to 20 wt.-%, or from 1 to 10 wt.-%, xanthan. In more specific embodiments, the synergistic fibre composition, based on its total weight, comprises, or consists of, from 36 to 40 wt.-% hydroxypropylmethylcellulose (HPMC), from 36 to 40 wt.-% glycerolmonostearate (GMS), from 17 to 21 wt.-%, glycerolmonooleate (GMO), from 1 to 3 wt.-%, lecithin, and from 1 to 5 wt.-% xanthan. In yet more specific embodiments, the synergistic fibre composition, based on its total weight, comprises, or consists of, from 36 to 40 wt.-% hydroxypropylmethylcellulose (HPMC), from 36 to 40 wt.-% glycerolmonostearate (GMS), from 17 to 21 wt.-%, glycerolmonooleate (GMO), from 1 to 3 wt.-%, lecithin, and from 1 to 5 wt.-% xanthan; and the xanthan is provided either in the form of a powder top-coating layer on the synergistic fibre composition and/or embedded in the lipids together with the HPMC.

In specific embodiments, the synergistic fibre composition may be provided in any suitable form that allows for, and is suitable for, oral administration; preferably as a solid dosage form. Examples of suitable solid dosage forms of the synergistic fibre composition include solid dosage forms such as tablets, capsules (two-piece hard capsules, single-piece soft capsules), minitablets, granules, pellets (i.e. granules of essentially roun(ed) shapes; e.g. spheronized granules), and powders. In one of the preferred embodiments, the synergistic fibre composition is provided in the form of a plurality of dry, flowable, ingestible particles, for instance as minitablets, granules, pellets, or powders. In specific embodiments, said ingestible particles exhibit a sieve diameter above 0.01 mm, or above 0.05 mm, or above 0.1 mm, or above 0.2 mm, or above 0.3 mm. In further specific embodiments, said ingestible particles exhibit a sieve diameter up to 3.0 mm, or up to 2.0 mm, or up to 1.5 mm, or up to 1.0 mm, or up to 0.8 mm, or up to 0.6 mm, or up to 0.5 mm. In more specific embodiments, said ingestible particles exhibit a sieve diameter in the range of 0.01 mm-3.0 mm, or 0.1 mm-3.0 mm, or 0.2 mm-3.0 mm, or 0.5 mm-3.0 mm, or 0.1 mm-2.5 mm, or 0.2 mm-2.5 mm, or 0.5 mm-2.5 mm, or 0.1 mm-2.0 mm, or 0.2 mm-2.0 mm, or 0.5 mm-2.0 mm, or 0.1 mm-1.5 mm, or 0.2 mm-1.5 mm, or 0.5 mm-1.5 mm, or 0.1 mm-1.25 mm, or 0.2 mm-1.25 mm, or 0.5 mm-1.25 mm, or 0.1 mm-1.0 mm, or 0.2 mm-1.0 mm, or 0.5 mm-1.0 mm, or 0.1 mm-0.8 mm, or 0.2 mm-0.8 mm, 0.5 mm-0.8 mm. Preferably, the ingestible particles exhibit a sieve diameter in the range of 0.1 mm-1.25 mm, such as 0.2 mm-1.0 mm, 0.2 mm-0.5 mm, or 0.5 mm-1.25 mm, or 0.5 mm-1.25 mm.

Since the synergistic fibre composition according to the above preferred embodiments comprises a plurality of these ingestible particles, the particle sizes described above in terms of sieve diameters should be interpreted to characterise the mass median sieve diameter of said plurality of ingestible particles; or, in other words, at least the mass median sieve diameters should comply with the above particle size provisions, e.g. falling within the above range of 0.01 mm-3.0 mm. Of course, it is preferred, that a majority of the ingestible particles complies with the above particle size provisions; for instance, at least 75 wt.-%, preferably at least 85 wt.-%, more preferably at least 95 wt.-% of the ingestible particles.

Moreover, since the synergistic fibre composition according to the first aspect of the invention is typically administered, or prepared for ingestion, by dispersion in water or another ingestible, aqueous medium, and only starts to dissolve after a lag time (i.e. typically after ingestion), it is preferred that the ingestible particles, or at least the above-described majority thereof, exhibit a particle size up to 1.25 mm, preferably up to 1.0 mm, not larger, so as to prevent or reduce any foreign object sensations in the mouth, or on the tongue. Furthermore, nanoparticles or particles with sizes below 0.01 mm, or particles larger than 3.0 mm are typically less preferred in terms of handling; for instance, they may not flow properly from the storage container and/or be less convenient for precise dosing.

As mentioned above, according to the present invention, the dietary fibre is the active ingredient of the synergistic fibre composition in the sense that the dietary fibre is administered purposefully to achieve a beneficial effect in the body after oral administration of said dietary fibre; for instance, deleayed gastric emptying and delayed nutrient absorption due to increased viscosity of the gastrointestinal contents, in particular the chyme, by a viscous fibre. Hence, in specific embodiments, the synergistic fibre composition is provided in the form of a plurality of dry, flowable, ingestible particles, for instance as minitablets, granules, pellets, or powders, and said ingestible particles are free of a synthetic drug substance.

In specific embodiments, the viscosity, that is achieved by mixing the synergistic fibre composition according to the first aspect of the invention in an aqueous medium, is measured at a temperature in the range of from 15 to 40° C. In more specific embodiments, the viscosity in the aqueous medium is measured at room temperature, or at a temperature of about 37° C. In yet more specific embodiments, the viscosity in the aqueous medium is measured at room temperature, or at a temperature of about 37° C., and the aqueous medium is selected from water, fasted-state simulated intestinal fluid (FaSSIF), fed-state simulated intestinal fluid (FeSSIF), fasted-state simulated intestinal fluid (FaSSGF), 0.1 N HCl, pH 6.8 phosphate-buffer solution, or similar bio-relevant media.

In specific embodiments, η_{max_syn} (i.e. the maximum viscosity that is achieved by mixing the synergistic fibre composition, comprising the dietary fibre(s) at an amount x_{_fibre}, in a given volume Vₓ of the aqueous medium) is at least 10%, preferably at least 15%, more preferably at least 20%, higher than η_{max_Σ} (i.e. the cumulative maximum viscosity of η_{max_fibre} plus η_{max_other}) in water at room temperature. In more specific embodiments, the synergistic fibre composition further comprises xanthan; and η_{max_syn} is at least 30%, preferably at least 40%, more preferably at least 50% higher than η_{max_Σ} in water at room temperature. In yet more specific embodiments, the synergistic fibre composition further comprises xanthan in the form of a powder top-coating layer, at a coating level in the range of 1 to 20 wt.-%, or 1 to 10 wt-%, or 2 to 8 wt.-%, based on the total weight of the uncoated cores; and η_{max_syn} is at least 30%, preferably at least 40%, more preferably at least 50% higher than η_{max_Σ} in water at room temperature.

In further specific embodiments, the swelling exhibits sigmoidal viscosity kinetics characterized by a lag time of at least 20 min, followed by a viscosity increase to at least 90% of the maximum viscosity η_{max_syn}, within no more than 2 h. In more specific embodiments, the swelling behavior is modified in such a way that in water at room temperature:
η_{max_syn} is at least 10%, preferably at least 15%, more preferably at least 20%, higher than η_{max_Σ}; and
the swelling exhibits sigmoidal viscosity kinetics characterized by a lag time of at least 20 min, followed by a viscosity increase to at least 90% of the maximum viscosity (η_{max_syn}), that is achieved by mixing the synergistic fibre composition, comprising the dietary fibre(s) at an amount x_{_fibre}, in a given volume Vₓ of the water, within no more than 2 h.

In yet further specific embodiments, the swelling exhibits sigmoidal viscosity kinetics characterized by a lag time of at least 25 min, followed by a viscosity increase to at least 70% of the maximum viscosity η_{max_syn}, within no more than 90 min. In more specific embodiments, the swelling behavior is modified in such a way that in water at room temperature:
η_{max_syn} is at least 10%, preferably at least 15%, more preferably at least 20%, higher than η_{max_Σ}; and
the swelling exhibits sigmoidal viscosity kinetics characterized by a lag time of at least 25 min, followed by a viscosity increase to at least 70% of the maximum viscosity (η_{max_syn}), that is achieved by mixing the synergistic fibre composition, comprising the dietary fibre(s) at an amount x_{_fibre}, in a given volume Vₓ of the water, within no more than 90 min.

The synergistic fibre composition according to the present invention may inter alia be used for the treatment and/or prevention of pre-diabetes and diabetes, especially diabetes type 2 (e.g. by levelling out post-prandial ‘blood sugar spikes’); hence, in one of the further preferred embodiments, the synergistic fibre composition is substantially free of sugars such as sucrose.

Furthermore, since the synergistic fibre composition according to the present invention is intended for oral administration, its inherent taste is important, too; especially in the preferable absence of taste-masking substances such as sucrose. Therefore, the use of poorly tasting lipids such as glycerolmonolaurate (GML) or lauric acid in the synergistic fibre composition should be limited to less than 20 wt.-%, preferably less than 10 wt.-%, based on the total weight of the composition. Optionally, the synergistic fibre composition according to the present invention is substantially free of glycerolmonolaurate (GML) or lauric acid.

In a second aspect, the present invention provides a method of modifying the swelling behavior of a dietary fibre in an aqueous medium by providing a synergistic fibre composition comprising (a) the dietary fibre, and (b) a lipid material comprising a lipid selected from (bi) a free fatty acid exhibiting a melting point in the range of from 30 to 60° C., (b₂) a glycerol- or sorbitan based fatty acid ester exhibiting a melting point in the range of from 30 to 75° C., (b₃) a plant-derived wax exhibiting a melting point in the range of from 30 to 75° C., or a homogenous mixture of two or more of the lipids under bi, b₂ and/or b₃, wherein the homogenous mixture of these lipids exhibits a melting point in the range of from 30 to 70° C.; wherein the dietary fibre is embedded within the lipid material, and wherein the swelling behavior is modified in such a way that:
the maximum viscosity that is achieved by mixing the synergistic fibre composition, comprising the dietary fibre(s) at an amount x_{_fibre}, in a given volume Vₓ of the aqueous medium (is higher than the maximum viscosity achieved in the same volume Vₓ of the aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}), and higher than the cumulative maximum viscosity of η_{max_fibre} plus η_{max_other} (η_{max_Σ}); and optionally
the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.

In one of the preferred embodiments of said method, the synergistic fibre composition provided for the method exhibits both these viscosity modifying features; i.e. the swelling behavior is modified in such a way that:
η_{max_syn} is higher than η_{max_fibre}, higher than η_{max_other}, and higher η_{max_Σ} (i.e. η_{max_fibre}+η_{max_other}); and
the swelling exhibits sigmoidal viscosity kinetics characterized by a lag time of at least 15 min, followed by a viscosity increase to at least 80% of η_{max_syn} within no more than 2 h.

In specific embodiments, the weight ratio of the dietary fibre under (a) to the lipid material under (b) in the synergistic fibre composition provided for the method is in the range from 30:70 to 60:40, or from 33:67 to 55:45, or from 35:65 to 50:50, or from 35:65 to 45:55, or from 37:63 to 42:58, preferably from 35:65 to 50:50.

In further specific embodiments, the synergistic fibre composition provided for the method comprises at least 45 wt.-%, or at least 50 wt.-%, or at least 55 wt.-%, or at least 60 wt.-% of the lipid material under (b) based on the total weight of the synergistic fibre composition.

In yet further specific embodiments, the synergistic fibre composition provided for the method comprises at least 50 wt.-% of the lipid material under (b) based on the total weight of the synergistic fibre composition; and the weight ratio of the dietary fibre under (a) to the lipid material under (b) in the synergistic fibre composition is in the range from 35:65 to 50:50.

In specific embodiments of the method, the dietary fibre under (a) is melt-embedded within, optionally melt-extruded with, the lipid material under (b).

In further specific embodiments of the method, the dietary fibre is a so-called viscous fibre, or high-viscosity fibre. For instance, in specific embodiments of the method, the dietary fibre is a water-soluble fibre, optionally a water-soluble fibre selected from cellulose ethers, and/or natural gums; optionally uncharged natural gums. In more specific embodiments of the method, the dietary fibre is a water-soluble cellulose ether selected from the group consisting of hydroxpropylmethylcellulose (HPMC), methylcellulose (MC), and carboxymethyl-cellulose (CMC); and/or a water-soluble, uncharged natural gum selected from the group consisting of psyllium seed husk, xanthan gum, galactomannan (e.g. from guar gum or locust bean gum), and glucomannan (e.g. from konjac gum).

In specific embodiments, the synergistic fibre composition provided for the method does not comprise any dietary fibres other than a water-soluble cellulose ether selected from the group consisting of hydroxpropylmethylcellulose (HPMC), methylcellulose (MC), and carboxymethylcellulose (CMC); or a water-soluble, uncharged natural gum selected from the group consisting of psyllium seed husk, xanthan gum, galactomannan, and glucomannan.

In further specific embodiments, the synergistic fibre composition provided for the method is substantially free of poly(carboxylates), poly(methacrylic acid), copolymers of acrylic and methacrylic acid, poly(hydroxyethyl methacrylic acid), alginic acid or salts thereof, microcrystalline cellulose, chitosan, gum arabic, beta glucan and pectins.

In specific embodiments of the method, the lipid material under (b) comprises at least one lipid selected from the group consisting of monoglycerides, triglycerides, and sorbitan-fatty acid triesters. In more specific embodiments, the lipid material under (b) comprises at least one lipid selected from the group consisting of monoglycerides, triglycerides, and sorbitan-fatty acid triesters; and the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, of said selected lipid(s), based on the total weight of the lipid material (b).

In further specific embodiments of the method, the lipid material under (b₂) is selected from the group consisting of glycerolmonostearate (GMS), glycerolmonooleate (GMO), tristearin (TS), and a triglyceride-based hard fat (Adeps solidus).

In yet further specific embodiments of the method, the lipid material under (b₂) is a triglyceride-based hard fat exhibiting a melting point in the range of 30 to 45° C. and/or a hydroxylvalue values of 5-50; for instance, a triglyceride-based hard fat exhibiting a melting point in the range of 40 to 45° C. and/or a hydroxylvalue values of 5-15; and/or a triglyceride-based hard fat exhibiting a melting point in the range of 32 to 37° C. and/or a hydroxylvalue values of 20-30. In more specific embodiments of the method, the lipid material under (b₂) is a triglyceride-based hard fat exhibiting a melting point in the range of 40 to 45° C. and a hydroxylvalue values of 5-15; and/or a triglyceride-based hard fat exhibiting a melting point in the range of 32 to 37° C. and a hydroxylvalue values of 20-30.

In yet further specific embodiments of the method, the lipid material under (b₂) is selected from the group consisting of glycerolmonostearate (GMS), glycerolmonooleate (GMO), tristearin (TS), and a triglyceride-based hard fat; and the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, of said selected lipid(s), based on the total weight of the lipid material (b).

In specific embodiments of the method, the lipid material under (b) further comprises an additional lipid (b′) in a homogenous mixture with the one or more lipid(s) under bi, b₂ and/or b₃, wherein additional lipid (b′) is selected from the group consisting of glycerol, a phosphatidylcholine, a sorbitan mono fatty acid ester, one or more fatty acid ester(s) selected from the group consisting of fatty acid esters of acetic acid, lactic acid, citric acid, tartaric acid, monoacetyl tartaric acid and diacetyl tartaric acid, one or more fatty acids with a melting point below 30° C.; and the homogenous mixture exhibits a melting point in the range of from 30 to 70° C. In more specific embodiments, the additional lipid (b′) comprised in the lipid material under (b) in a homogenous mixture with the one or more lipid(s) under bi, b₂ and/or b₃, is a phosphatidylcholine, optionally a lecithin such as egg lecithin, soy lecithin, or sunflower lecithin. Alternatively, or in addition to the phosphatidylcholine, the additional lipid (b′) comprised in the lipid material under (b) in a homogenous mixture with the one or more lipid(s) under b₁, b₂ and/or b₃, is a sorbitan mono fatty acid ester, optionally a sorbitan mono fatty acid ester selected from the group consisting of sorbitanmonooleate, polyoxyethylene sorbitanmonolaurate, polyoxyethylen sorbitanmonostearate, and polyoxyethylen sorbitanmonooleate.

In specific embodiments of the method, the lipid material under (b₁) is selected from the group consisting of saturated fatty acids, C₈-C₁₄ fatty acids, and saturated C₈-C₁₄ fatty acids. In more specific embodiments, the lipid material under (b₁) is selected from the group consisting of myristic acid and capric acid.

In further specific embodiments of the method, the lipid material under (b₃) is a plant-derived wax selected from the group consisting of candelilla wax, carnauba wax, berry wax, myrica fruit wax, soy wax, rice bran wax, and bees wax. In more specific embodiments, the lipid material under (b₃) is candelilla wax.

In specific embodiments of the method, the lipid material under (b) comprises at least 70 wt.-%, or at least 75 wt.-%, or at least 80 wt.-%, or at least 85 wt.-%, or at least 90 wt.-%, of the lipids (b₁) to (b₃) based on the total weight of the lipid material (b).

In more specific embodiments, the lipid material under (b) consists of the lipids (b₁) to (b₃), or optionally of the lipids (b₁) to (b₃) and the additional lipids (b′).

In specific embodiments of the method, the lipid material under (b) exhibits a melting point in the range of from 30 to 70° C.

In one of the preferred embodiments of the method, the lipid material under (b) comprises at least one lipid selected from the group consisting of myristic acid, capric acid, glycerolmonostearate (GMS), tristearin, and candelilla wax. In more specific embodiments of the method, the lipid material under (b) comprises at least one lipid selected from the group consisting of myristic acid, capric acid, glycerolmonostearate (GMS), tristearin, and candelilla wax; and the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b). In yet more specific embodiments of the method, the lipid material under (b) comprises at least one lipid selected from the group consisting of myristic acid, capric acid, glycerolmonostearate (GMS), tristearin, and candelilla wax; the lipid material under (b) comprises at least 50 wt-%, or at least 55 wt.-%, or at least 60 wt.-%, or at least 65 wt.-%, or at least 70 wt-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b); and the lipid material under (b) comprises at least one other lipid selected from the group consisting of caprylic acid, glycerol, and glycerolmonooleate (GMO). In yet more specific embodiments of the method, the lipid material under (b) comprises at least one lipid selected from the group consisting of myristic acid, capric acid, glycerolmonostearate (GMS), tristearin, and candelilla wax; the lipid material under (b) comprises at least 50 wt.-%, or at least 55 wt.-%, or at least 60 wt-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b); the lipid material under (b) comprises at least one other lipid selected from the group consisting of caprylic acid, glycerol, and glycerolmonooleate (GMO); and wherein the lipid material under (b) further comprises lecithin.

In specific embodiments of the method, the synergistic fibre composition further comprises xanthan, either in the form of a powder top-coating layer, and/or with the xanthan being embedded in the lipid(s) under (b) together with the dietary fibre under (a). In a more specific embodiment, the synergistic fibre composition further comprises xanthan and the further dietary fibre under (a) in a weight ratio of fibre to xanthan in the range of 98:2 to 75:25, 96:4 to 80:20, or 94:6 to 82:18; such as 95:5, 91:9 or 85:15. In a yet more specific embodiment, the synergistic fibre composition further comprises xanthan in the form of a powder top-coating layer, and the coating level thereof is in the range of 1 to 20 wt.-%, or 1 to 10 wt.-%, or 2 to 8 wt.-%, based on the total weight of the uncoated cores.

In one of the preferred embodiments, the synergistic fibre composition provided for the method comprises, or consists of, hydroxpropylmethylcellulose (HPMC) as the dietary fibre under (a); and glycerolmonostearate (GMS), glycerolmonooleate (GMO), and lecithin as the components of the lipid material under (b). In more specific embodiments, the synergistic fibre composition provided for the method comprises, or consists of, hydroxypropylmethylcellulose (HPMC) and xanthan as the dietary fibres under (a); and glycerolmonostearate (GMS), glycerolmonooleate (GMO), and lecithin as the components of the lipid material under (b). In yet more specific embodiments, the synergistic fibre composition provided for the method comprises, or consists of, hydroxypropylmethyl-cellulose (HPMC) and xanthan as the dietary fibres under (a); glycerolmonostearate (GMS), glycerolmonooleate (GMO), and lecithin as the components of the lipid material under (b); and the xanthan is provided either in the form of a powder top-coating layer on the synergistic fibre composition, and/or embedded within the lipid material together with the hydroxypropyl-methylcellulose (HPMC). In yet more specific embodiments, the synergistic fibre composition provided for the method comprises, or consists of, hydroxypropylmethylcellulose (HPMC) and xanthan as the dietary fibres under (a); glycerolmonostearate (GMS), glycerolmonooleate (GMO), and lecithin as the components of the lipid material under (b); and the xanthan is provided in the form of a powder top-coating layer on the synergistic fibre composition.

All embodiments, including all specific or preferred embodiments, as described above in connection with the synergistic fibre composition of the first aspect of the invention also apply to the method of modifying the swelling behavior of a dietary fibre in an aqueous medium according to the second aspect of the invention.

For instance, in one of the preferred embodiment, the synergistic fibre composition provided for the method, based on its total weight, comprises, or consists of, from 25 to 55 wt.-%, or from 35 to 45 wt.-%, hydroxypropylmethylcellulose (HPMC), from 25 to 55 wt.-%, or from 35 to 45 wt.-%, glycerolmonostearate (GMS), from 10 to 30 wt.-%, or from 15 to 25 wt.-%, glycerolmonooleate (GMO), from 1 to 20 wt.-%, or from 1 to 10 wt.-%, lecithin, and from 1 to 20 wt.-%, or from 1 to 10 wt.-%, xanthan. In more specific embodiments, the synergistic fibre composition, based on its total weight, comprises, or consists of, from 36 to 40 wt.-% hydroxypropylmethylcellulose (HPMC), from 36 to 40 wt.-% glycerolmonostearate (GMS), from 17 to 21 wt.-%, glycerolmonooleate (GMO), from 1 to 3 wt.-%, lecithin, and from 1 to 5 wt.-% xanthan. In yet more specific embodiments, the synergistic fibre composition, based on its total weight, comprises, or consists of, from 36 to 40 wt.-% hydroxypropylmethylcellulose (HPMC), from 36 to 40 wt.-% glycerolmonostearate (GMS), from 17 to 21 wt.-%, glycerolmonooleate (GMO), from 1 to 3 wt.-%, lecithin, and from 1 to 5 wt.-% xanthan; and the xanthan is provided either in the form of a powder top-coating layer on the synergistic fibre composition and/or embedded in the lipids together with the HPMC.

As mentioned above, the synergistic fibre composition provided for the method may take any suitable form that allows for, and is suitable for, oral administration; preferably a solid dosage form such as tablets, capsules, minitablets, granules, pellets, and powders. In one of the preferred embodiments, the synergistic fibre composition is provided in the form of a plurality of dry, flowable, ingestible particles, for instance as minitablets, granules, pellets, or powders, said ingestible particles exhibiting a sieve diameter in the range of 0.01 mm-3.0 mm, or 0.1 mm-3.0 mm, or 0.2 mm-3.0 mm, or 0.5 mm-3.0 mm, or 0.1 mm-2.5 mm, or 0.2 mm-2.5 mm, or 0.5 mm-2.5 mm, or 0.1 mm-2.0 mm, or 0.2 mm-2.0 mm, or 0.5 mm-2.0 mm, or 0.1 mm-1.5 mm, or 0.2 mm-1.5 mm, or 0.5 mm-1.5 mm, or 0.1 mm-1.25 mm, or 0.2 mm-1.25 mm, or 0.5 mm-1.25 mm, or 0.1 mm-1.0 mm, or 0.2 mm-1.0 mm, or 0.5 mm-1.0 mm, or 0.1 mm-0.8 mm, or 0.2 mm-0.8 mm, 0.5 mm-0.8 mm. Preferably, the ingestible particles exhibit a sieve diameter in the range of 0.1 mm-1.25 mm, such as 0.2 mm-1.0 mm, 0.2 mm-0.5 mm, or 0.5 mm-1.25 mm, or 0.5 mm-1.25 mm.

In specific embodiments of the method according to the second aspect of the invention, the synergistic fibre composition is provided in the form of a plurality of dry, flowable, ingestible particles, for instance as minitablets, granules, pellets, or powders, and said ingestible particles are free of a synthetic drug substance.

In specific embodiments of the method according to the second aspect of the invention, the viscosity is measured at a temperature in the range of from 15 to 40° C. In more specific embodiments, the viscosity in the aqueous medium is measured at room temperature, or at a temperature of about 37° C. In yet more specific embodiments, the viscosity in the aqueous medium is measured at room temperature, or at a temperature of about 37° C., and the aqueous medium is selected from water, fasted-state simulated intestinal fluid (FaSSIF), fed-state simulated intestinal fluid (FeSSIF), fasted-state simulated intestinal fluid (FaSSGF), 0.1 N HCl, pH 6.8 phosphate-buffer solution, or similar bio-relevant media.

In specific embodiments, η_{max_syn} (i.e. the maximum viscosity that is achieved by mixing the synergistic fibre composition, comprising the dietary fibre(s) at an amount x_{_fibre}, in a given volume Vₓ of the aqueous medium) is at least 10%, preferably at least 15%, more preferably at least 20% higher, than η_{max_Σ} (the cumulative maximum viscosity of η_{max_fibre} plus η_{max_other}) in water at room temperature. In more specific embodiments, the synergistic fibre composition further comprises xanthan; and η_{max_syn} is at least 30%, preferably at least 40%, more preferably at least 50% higher than η_{max_Σ} in water at room temperature. In yet more specific embodiments, the synergistic fibre composition further comprises xanthan in the form of a powder top-coating layer, at a coating level in the range of 1 to 20 wt.-%, or 1 to 10 wt.-%, or 2 to 8 wt.-%, based on the total weight of the uncoated cores; and η_{max_syn} is at least 30%, preferably at least 40%, more preferably at least 50% higher than η_{max_Σ} in water at room temperature.

In further specific embodiments, the swelling exhibits sigmoidal viscosity kinetics characterized by a lag time of at least 20 min, followed by a viscosity increase to at least 90% of the maximum viscosity η_{max_syn}, within no more than 2 h. In more specific embodiments, the swelling behavior is modified in such a way that in water at room temperature:
η_{max_syn} is at least 10%, preferably at least 15%, more preferably at least 20%, higher than η_{max_Σ}; and
the swelling exhibits sigmoidal viscosity kinetics characterized by a lag time of at least 20 min, followed by a viscosity increase to at least 90% of the maximum viscosity (η_{max_syn}), that is achieved by mixing the synergistic fibre composition, comprising the dietary fibre(s) at an amount x_{_fibre}, in a given volume Vₓ of the water, within no more than 2 h.

In yet further specific embodiments, the swelling exhibits sigmoidal viscosity kinetics characterized by a lag time of at least 25 min, followed by a viscosity increase to at least 70% of the maximum viscosity η_{max_syn}, within no more than 90 min. In more specific embodiments, the swelling behavior is modified in such a way that in water at room temperature:
η_{max_syn} is at least 10%, preferably at least 15%, more preferably at least 20%, higher than η_{max_Σ}; and
the swelling exhibits sigmoidal viscosity kinetics characterized by a lag time of at least 25 min, followed by a viscosity increase to at least 70% of the maximum viscosity (η_{max_syn}), that is achieved by mixing the synergistic fibre composition, comprising the dietary fibre(s) at an amount x_{_fibre}, in a given volume Vₓ of the water, within no more than 90 min.

In a third aspect, the invention relates to a process for preparing the synergistic fibre composition provided for the method according to the second aspect of the invention, or specifically the synergistic fibre composition according to the first aspect of the invention, wherein the process comprises a step of processing a blend comprising the dietary fibre under (a) and the lipid material under (b) by:
(i) extruding, or melt-extruding, the blend;
(ii) spray-congealing the blend, optionally using a jet-break-up technique;
(iii) melt-granulating the blend;
(iv) compressing the blend into minitablets;
(v) melt-injecting the blend into a liquid medium; or
(vi) spray-coating the blend onto inert cores.

These processes are suitable in that they allow to prepare the synergistic fibre composition in such a way that the dietary fibre is embedded within the lipid materials as intended. Preferably, the final product of any one of the processes under (i)-(vi) is a homogenous matrix composition in which the lipid material forms a continuous, coherent phase in which the dietary fibre is discontinuous and homogenously dispersed. This means that in specific embodiments, the processes under (i)-(vi) may be preceded by a mixing step (0) in which the dietary fibre under (a) and the lipid material under (b) are first mixed together to form homogenous blend, prior to said blend being processed further; for instance, prior to compressing the blend into minitablets.

Further preferably, the synergistic fibre composition is prepared in such a way that the resulting processed blend also exhibits a greatly reduced porosity compared to, for instance, compositions obtained from mere dry-granulation of the dietary fibre, or wet-granulation thereof with an aqueous or hydroalcoholic binder solution. This is achieved by either melting the lipid material under (b) and homogenously dispersing the dietary fibre under (a) in the melt; or by applying sufficient pressure onto the blend to render the lipid material pliable and ‘fuse’ it around the dispersed (as is the case when extruding without heating or when compressing minitablets)

In one of the preferred embodiments, the dietary fibre under (a) is melt-embedded within, optionally melt-extruded with, the lipid material under (b). In a specific embodiment, the extrusion, or melt-extrusion, under (i) is carried out in twin-screw extruder.

Optionally, the process according to the third aspect of the invention may be finished by a top-coating step. In specific embodiments, the top-coating step is carried out to apply a powder top-coating layer. In further specific embodiments, the top-coating step is carried out to apply a powder top-coating layer of xanthan.

All embodiments, including all specific or preferred embodiments, as described above in connection with the synergistic fibre composition provided for the method according to the second aspect of the invention, or specifically the synergistic fibre composition according to the first aspect of the invention, also apply to the process for preparing said synergistic fibre composition(s) according to the third aspect of the invention.

In a fourth aspect, the invention relates to the synergistic fibre composition provided for the method according to the second aspect of the invention, or specifically the synergistic fibre composition according to the first aspect of the invention, for use as a medicament.

In a fifth aspect, the invention relates to the synergistic fibre composition provided for the method according to the second aspect of the invention, or specifically the synergistic fibre composition according to the first aspect of the invention, for use in the treatment and/or prevention of gastro-intestinal and/or metabolic disorders. In specific embodiments thereof, the gastro-intestinal and/or metabolic disorder is selected from the group consisting of constipation, diverticulosis, irritable bowel syndrome and Crohn's disease, elevated plasma cholesterol levels, metabolic syndrome, pre-diabetes, diabetes, specifically, diabetes type 2, overweight, and obesity. These conditions benefit from administration of the synergistic fibre composition, in particular the synergistic fibre composition according to the first aspect of the invention for various reasons; for instance, conditions such as constipation, diverticulosis, irritable bowel syndrome and Crohn's disease benefit from its good water-binding capacity in the large bowel; conditions such as elevated plasma cholesterol levels and metabolic syndrome from the decreased reuptake of bile acids and cholesterol which ultimately lowers blood levels of LDL-cholesterol; and conditions such as prediabetes, diabetes, specifically diabetes type 2, overweight, or obesity benefit from the increased viscosity of the gastrointestinal contents that allows for delayed gastric emptying and delayed nutrient uptake, both of which facilitate a longer lasting satiety and more stable blood sugar levels due to delayed glucose uptake.

In fact, the synergistic fibre composition(s) of the present invention can be employed for all indications, specifically medical indications, for which the dietary fibre contained therein is already used for with respective prior art products; however, with the added benefit that either less fibre is required for the same viscosity effect, and/or that the fibre is more conveniently ingestible (e.g. without forming lumps upon dispersion of the composition in an aqueous medium, and/or without feeling ‘slimy’ or too thick upon ingestion).

Hence, in a sixth aspect of the invention, the invention relates to a solid dietary fibre formulation for oral administration comprising, or consisting of, the synergistic fibre composition provided for the method according to the second aspect of the invention, or specifically the synergistic fibre composition according to the first aspect of the invention, wherein the synergistic fibre composition is provided in the form of a plurality of dry, flowable, ingestible particles, for instance as minitablets, granules, pellets, or powders, or has been prepared from said plurality of dry, flowable, ingestible particles. For instance, the plurality of dry, flowable, ingestible particles may be compressed into tablets, or filled into capsules, sachets, stick packs, or other containers (e.g. bottles or vials made of glass or plastic materials). In specific embodiments, the particles are filled into sachets, stick packs, or other containers, in such a way that each single dose is accommodated in one primary package.

In further specific embodiments, the solid dietary fibre formulation exhibits a high content of the dry, flowable, ingestible particles, such as at least 50 wt.-%, or at least 60 wt.-%, or at least 70 wt.-%, or at least 80 wt.-%, or at least 90 wt.-%, or at least 95 wt.-%, by weight.

Optionally, the solid dietary fibre formulation may comprise the particles along with one or more further inactive ingredients; for instance, flavourings such as sugars or sugar alcohols, sweeteners, aromas, anti-foaming agents. The expression ‘inactive ingredients’ in this regard means that the solid dietary fibre formulation is substantially free of both synthetic drug substances and further viscosity increasing substances (other than those provided with the particles of the synergistic fibre composition). Having health concerns and the intended use of the solid dietary fibre formulation in mind (e.g. the needs of diabetic, or pre-diabetic patients) the use of sugars such as sucrose in particular, and/or other insulin-triggering flavourings as part(s) of the inactive ingredients should be kept to a minimum. Optionally, both the solid dietary fibre formulation, and the solid dietary fibre composition comprised therein, are substantially free of sugars such as sucrose.

In specific embodiments, a single dose of the solid dietary fibre formulation comprises at least 0.5 g of the dietary fibre under (a), preferably at least 1 g thereof. In further specific embodiments, a single dose of the solid dietary fibre formulation comprises up to 15 g of the dietary fibre under (a). In yet further specific embodiments, a single dose of the solid dietary fibre formulation comprises from 0.5 g to 15 g of the dietary fibre under (a). Preferably, the single dose required for the intended effect of a specific dietary fibre, in particular a viscous fibre, is lower than with prior art compositions of the same fibre; for instance, in specific embodiments, the amount of the dietary fibre under (a) comprised in a single dose of the solid dietary fibre formulation is from 0.5 g to 10 g of the composition, or from 0.5 g to 8 g or 0.5 g to 6 g, or 0.5 g to 5 g, or from 0.5 g to 4 g, or 0.5 g to 2.5 g, or 0.5 g to 2 g respectively. Said single doses of the solid dietary fibre formulation may be administered once daily, or two to five times daily (e.g. 3 times or 4 times daily); optionally together with or about 30 min before a meal.

Again, all embodiments, including all specific or preferred embodiments, as described above in connection with the synergistic fibre composition provided for the method according to the second aspect of the invention, or specifically the synergistic fibre composition according to the first aspect of the invention, also apply to the use of said synergistic fibre composition(s) according to the fourth, fifth and sixth aspect of the invention.

### EXAMPLES

The following examples serve to illustrate the invention; however, they should not to be understood as restricting the scope of the invention.

### Example 1—Lab Scale Process for Preparing the Synergistic Fibre Composition (Melt-Granules)

Step 1: The lipid components were melted in a pot or beaker on a heating plate. Then, the viscous fibre powder was thoroughly stirred in, and the mixture subsequently poured into plastic bags and cooled at −20° C. in a freezer over night. The resulting solid mixture was ground in a blender/food processor (Kenwood Chef KVC3110S or Bosch MUM4880) and sieved so as to yield a granule fraction with a sieve diameter of about 0.5 to 1.25 mm.

Optionally, an aliquot of this granule fraction received a powder-layer of xanthan gum by physically mixing the melt-granuled fibre with xanthan powder (e.g. Xanthan FF, Jungbunzlauer, Switzerland), for instance, 30 g granules plus 2 g xanthan, and subsequent sieving so as to remove excess xanthan gum powder and yield a granule fraction with a sieve diameter of about 0.5 to 1.25 mm again.

### Example 2—Lab Scale Process for Preparing More Compacted the Synergistic Fibre Composition Using an Extruder (Melt-Extrudates Lab)

Step 1: The lipid components were melted in a pot or beaker on a heating plate. Then, the viscous fibre powder was thoroughly stirred in, and the mixture subsequently poured into plastic bags and cooled at −20° C. in a freezer over night. The resulting solid mixture was ground in a blender/food processor (Kenwood Chef KVC3110S or Bosch MUM4880).

Step 2: To compact the resulting melt-granule further, it was then fed via a volumetric dosing system (Dosimex® DO-50, Gabler GmbH & Co KG, Germany) into a twin screw extruder (Extruder DE-40/10, Gabler GmbH & Co KG, Germany) operating at a rotation speed of 40 rpm and a product temperature of about 33° C. to extrude strands of 0.5 mm diameter. The extruded strands were collected in plastic bags and cooled at −20° C. in a freezer over night. Subsequently, the resulting compacted solid mixture was again ground in a blender/food processor (Kenwood Chef KVC3110S or Bosch MUM4880) and sieved so as to yield the extrudate fraction with a sieve diameter of about 0.5 to 1.25 mm.

Optionally, an aliquot of this granule fraction received a powder-layer of xanthan gum by physically mixing the melt-granuled fibre with xanthan powder (e.g. Xanthan FF, Jungbunzlauer, Switzerland), for instance, 30 g granules plus 2 g xanthan, and subsequent sieving so as to remove excess xanthan gum powder and yield a granule fraction with a sieve diameter of about 0.5 to 1.25 mm again.

### Example 3—Production Scale Process for Preparing More Compacted the Synergistic Fibre Composition Using an Extruder (Melt-Extrudates Production)

Example 3 describes a process for preparing melt-extrudates at production scale for an exemplary composition. A granule premix was prepared by filling 15.6 kg glycerol monostearate (GMS 90 food, Mosselman, Belgium), 7.8 kg glycerol monooleate (Imwitor® 990, Nordmann Rassmann GmbH, Germany) and 0.78 kg lecithin (SternPur SP, Sternchemie GmbH & Co. KG, Germany) into the mixing chamber of a granulation device (F130D, Gebrüder Lödige Maschinenbau GmbH, Germany) with the mixing tool running at a speed of 30 rpm and the chamber being heated using an external temperature control system (Compact TKN-90-18-35, Single Temperiertechnik GmbH, Germany) so as to yield a homogenous melt of the lipid components at 70° C. Then, 15.6 kg of hydroxypropylmethylcellulose (HPMC, AnyAddy® CN10T, Harke Pharma GmbH, Germany) was added to the homogenous melt of the lipid components and thoroughly blended in at 40 rpm until homogeneity, before turning off the heating system, rapidly adding about 11 kg of dry ice to the mixing chamber with the mixer running at 40 rpm and the choppers turned on. The resulting granule premix was released through the outlet and collected in bags for further processing.

The granule premix was fed via a volumetric dosing system (Dosimex® DO-50, Gabler GmbH & Co KG, Germany) into a twin screw extruder (Extruder DE-40/10, Gabler GmbH & Co KG, Germany) operating at a rotational speed of 40 rpm and a product temperature of about 33° C. to extrude strands of 0.5 mm diameter. The extruded strands were then filled again into the mixing chamber of the F130D-granulation device, in aliquots of 10 kg, and cut into smaller particles by first running the mixer at 40 rpm for 60 s, before rapidly adding 1.5 kg dry ice and turning on the choppers for 3 min to obtain particles of about 0.5-3.0 mm length.

Optionally, the resulting chopped particles subsequently received a powder-layer of xanthan by adding xanthan powder (e.g. Xanthan FF, Jungbunzlauer, Switzerland) into the mixing chamber of the granulation device after the ‘chopping step; for instance, 1 kg xanthan per 10 kg extruded strands. Subsequently, the particles (with or without the xanthan powder-layer) were classified on a sieving machine (Siftomat® 1, Fuchs Maschinen AG, Switzerland) so as to yield the 0.5 mm-1.0 mm granule fraction and to remove excess powder.

### Example 4/Viscosity Measurements

Protocol A—Maximum Viscosity in Water at Room Temperature

A volume of 600 mL tap-water of about 20° C. were provided in a beaker and stirred at room temperature (20±5° C.) using a heatable magnetic stirrer at 300 rpm. Pre-weighed amounts of the materials to be tested were poured into the beaker under stirring. As soon as all weighed material was added to the water, the mixture was stirred once, briefly but thoroughly, with a spoon to aid dispersion of the material. The resulting mixture, or slurry, was subsequently homogenized using a immersion blender (single speed household appliance, 175 W; OSB103W from OK). Afterwards, the mixture was kept at room temperature over night (without further stirring or agitation) to ensure complete dissolution of the materials, in particular of the viscous fibres, and to allow for removal of visible air bubbles from the fibre solution.

On the next day, the maximum viscosity of the clear or slightly opalescent, substantially bubble-free fibre solution was measured using a rotational spindle viscosimeter (Elcometer 23RV von Elcometer Limited) according to the manufacturer's protocol with the appropriate L1 or L2 spindle submerged completely into the fibre solution.

Protocol B—Viscosity Kinetics and Maximum in Water at Room Temperature

The measurements were performed same as in Protocol A with the difference that the immersion blender homogenization step was omitted to measure the viscosity kinetics (i.e. development of viscosity over time). Instead, a first viscosity measurement according to the manufacturer's protocol was already performed at time t=0 (t₀), once the complete, pre-weighed amount of the material(s) to be tested was added to the beaker and dispersed in the tap-water with a spoon. This was followed by further measurements every 5 minutes or every 15 minutes for a period of 2 hours (depending on how quickly the viscosity increase occurred).

After the measurement at 2 hours (t₂ₕ), the solution was kept over night at room temperature while stirring at 300 rpm using a heatable magnetic stirrer to ensure complete dissolution of the materials, in particular of the viscous fibres. Then two final measurements at 23.5 hours (t_{23.5h}) and 24 hours (t₂₄ₕ) were performed. The resulting data can be displayed in graphs showing viscosity (mPas) over time (e.g. in minutes), with the maximum viscosity, as well as time to reach ¼ or ½ of said maximum viscosity (t_{1/4}) and (t_{1/2}) being determined from this graph.

Protocol C—Viscosity Kinetics and Maximum in FaSSGF at 37° C.

The measurements and data analysis were performed same as in Protocol B but using 600 mL fasted state, simulated gastric fluid (FaSSGF) as the medium and keeping the temperature at 37° C. throughout the measurements (instead of tap-water at room temperature).

Viscosity measurements at 37° C. according to the manufacturer's protocol were performed at the same time points as in Protocol B, i.e. at to, then every 5 or 15 minutes until t₂ₕ, and then again at t_{23.5h} and t_{24 h} after over night shaking at 150 rpm and 37° C. using an Incubating Mini Shaker (VWR International).

The following Examples 5 to 10 and Tables 1 to 6, show an overview of various tested compositions, including synergistic fibre compositions according to the invention, prepared according to the processes of Examples 1 (granules lab scale), 2 (extrudates lab scale) or 3 (extrudates production scale) as indicated by the respective number 1-3 in the third column of the tables, along with their determined maximum viscosities (ηₘₐₓ), t_{1/4}, t_{1/2}, and the %-ratio of t_{1/4} to t_{1/2}. The weight-percentages of the components are indicated in parentheses.

These compositions were employed for testing viscosity kinetics and maximum viscosity in water or fasted-state simulated gastric fluid (FaSSGF) according to Example 4 Protocols A-C, as indicated by the respective letter A-C in the fourth column of the tables. All compositions have been prepared by the processes described in Examples 1 to 3 unless where expressly mentioned otherwise (e.g. pure fibre powders or dry powder mixtures); as indicated by “-” instead of a number 1-3 in the third column.

The following components and abbreviations and components were used: Viscous fibre components

HPMC (hydroxypropylmethylcellulose, AnyAddy® CN10T, Harke Pharma GmbH, Germany); HPMC K250M (hydroxypropylmethylcellulose, Metocel® K250M, Brenntag GmbH, Germany); X or Xanthan (xanthan gum; Xanthan FF, Jungbunzlauer, Switzerland); Psyllium (psyllium seed husks from plantago ovata, Carepsyllium 99/100, 100 mesh powder, Caremoli S.p.A. Italy); KGLM (konjac glucomannan powder, Harke Services GmbH, Germany); CMC (carboxymethylcellulose, Walocelm CRT 50000 PA07, Brenntag GmbH, Germany); Guar (raw guar gum from guar beans, Guar gum 5000 cP, Würzteufel GmbH, Germany)

### Lipid Components

GMS (glycerol monostearate, high-melting monoglyceride emulsifier, M_{P} 71-72° C., GMS 90 food, Mosselman, Belgium); GMO (glycerol monooleate; low-melting monoglyceride emulsifier, M_{P} 30° C., Imwitor® 990, Nordmann Rassmann GmbH, Germany); Liquid GMO (liquid glycerol monooleate, low-melting monoglyceride emulsifier, M_{P} 25° C., Glycerol monooleate 40% HO EP, Mosselman, Belgium); E85 (Witepsol® E85, high-melting triglyceride based hard fat (Adeps solidus), M_{P} 44° C., Nordmann Rassmann GmbH, Germany); W25 (Witepsol® W25, low-melting triglyceride based hard fat (Adeps solidus), M_{P} 36° C., Nordmann Rassmann GmbH, Germany); LCT (Sunflower derived lecithin, SternPur SP, Stern-Chemie GmbH & Co. KG, Germany); IWT372 (C₁₆-C₁₈ glycerides and salts, flakes, M_{P} 60° C., Imwitor® 372, IOI Oleo GmbH, Germany); Glycerol (glycerol, M_{P} 20° C., Sigma-Aldrich Chemie GmbH, Germany); TS (tristearin; Dynasan® 118, M_{P} 72-73° C., Nordmann Rassmann GmbH, Germany); Span 65 (sorbitan tristearate, M_{P} 53° C., Span® 65, Sigma-Aldrich Chemie GmbH, Germany); Span 80 (Sorbitan monooleate, viscous liquid, Span® 80, Sigma-Aldrich Chemie GmbH, Germany); Tween (polyoxyethylene sorbitan monooleate, Polysorbat 80, viscous liquid, M_{P}−21° C., Tween® 80, Sigma-Aldrich Chemie GmbH, Germany); caprylic (caprylic acid, M_{P} 16° C.; TCI Deutschland GmbH, Germany); capric and myristic (capric acid and myristic acid powders, M_{P} 32° C. and M_{P} 53° C.; Sigma-Aldrich Chemie GmbH, Germany); Candelilla (natural plant wax from euphorbia antisyphilitica, M_{P} 69-70° C.; Dragonspice Naturwaren, Germany);

For any of the tests shown in Examples 5 to 10 and Tables 1 to 6 below, the amounts were chosen in such a way as to be comparable; for instance, if the amount of the synergistic fibre composition tested contained 4 g fibre, then the comparative test with ‘fibre only’ was done with 4 g as well. Same applies to the amounts of the lipids, and, where applicable, the xanthan top-coating.

Furthermore, in order to describe the sigmoidal course of graphs representing the viscosity kinetics data, the time points of reaching one fourth and half of the maximum viscosity (t_{1/4} and t_{1/2}, respectively) were determined as illustrative parameters indicating if a graph describes a long or short lag-phase. The two times were also put in correlation (%-ratio of t_{1/4} to t_{1/2}) as an illustrative parameter if the viscosity increase describes a fast or slow disintegration of the fibre-composition and dissolution of the fibre therein; the higher the ratio, the faster.

### Example 5—Synergistic Effect of a GMS/GMO Mixture on Viscosity of Various Fibres

Table 1 below shows the synergistic effects of melt-granulating various viscous fibre with a 2:1 mixture of glycerol monostearate and glycerol monooleate (GMS/GMO), namely two different types of hydroxypropylmethylcellulose (HPMC; HPMC K250M), psyllium seed husk, konjac glucomannan (KGLM), carboxymethylcellulose (CMC), and guar gum.

TABLE 1

| | Composition (prepared acc. to | | | ηₘₐₓ | t_{1/2} | t_{1/4} | t_{1/4}/t_{1/2} |
|---|---|---|---|---|---|---|---|
| Nr. | Ex. 1-3, tested acc. to Ex. 4A-C) | | | [mPas] | [min] | [min] | [%] |
| 1 | HPMC powder (100) | — | A | 920 | — | — | — |
| 1 | HPMC powder (100) | — | B | 920 | 180 | 90 | 50 |
| 94 | HPMC K250M powder (100) | — | A | 1203 | | | |
| 7 | GMS/GMO lipid granules | 1 | A | 5 | — | — | — |
| | (66.7/33.3) | | | | | | |
| 5 | HPMC/GMS/GMO | 1 | B | 1215 | 62 | 47 | 76 |
| | (40/40/20) | | | | | | |
| 57 | HPMC K250M/GMS/GMO | 1 | B | 1665 | 62 | 51 | 82 |
| | (40/40/20) | | | | | | |
| 29 | Psyllium powder (100) | — | A | 84 | — | — | — |
| 31 | Psyllium/GMS/GMO | 1 | B | 212 | 53 | 31 | 58 |
| | (40/40/20) | | | | | | |
| 34 | KGLM powder (100) | — | B | 290 | 105 | 40 | 38 |
| 36 | KGLM/GMS/GMO | 1 | B | 442 | 20 | 15 | 75 |
| | (40/40/20) | | | | | | |
| 37 | CMC powder (100) | — | A | 807 | — | — | — |
| 39 | CMC/GMS/GMO | 1 | B | 1981 | 9 | 3 | 33 |
| | (40/40/20) | | | | | | |
| 40 | Guar powder (100) | — | A | 256 | — | — | — |
| 42 | Guar/GMS/GMO | 1 | B | 336 | 115 | 80 | 70 |
| | (40/40/20) | | | | | | |

Table 1 shows that, as expected, the GMS/GMO lipid mixture alone (Nr. 7) does not exhibit any noteworthy increase in viscosity, with a maximum viscosity of only 5 mPas. This is not surprising in so far as these lipids are not known to noticeably increase the viscosity of aqueous media such as water. Hence, the viscosity increase is expected to come predominantly as the result of the presence of the viscous fibre. As can be seen from Table 1, all tested viscous fibres surprisingly exhibit a synergistic increase in their maximum viscosity (ηₘₐₓ) when melt-granulated with the 2:1 GMS/GMO lipid mixture; or, in other words, their η_{max_syn} is higher than the maximum viscosity achieved by the various dietary fibres alone (η_{max_fibre}), higher than the maximum viscosity achieved by the GMS/GMO (η_{max_other}), and higher than the cumulative maximum viscosities of fibre alone plus GMS/GMO.

For instance, 4 g of pure HPMC powder (Nr. 1) in 600 mL water yielded a maximum viscosity (η_{max_fibre}) at room temperature of 920 mPas, and 6 g of a 2:1 mixture of GMS/GMO (Nr. 7) under same conditions yielded a maximum viscosity (η_{max_other}/η_{max_lipid}) of only 5 mPas; i.e. in sum 925 mPas. However, 10 g of the respective HPMC/GMS/GMO (40/40/20)-composition (Nr. 5) yielded a maximum viscosity (η_{max_syn}) of 1215 mPas in water at room temperature, or, in other words, a synergistic increase of over 30%.

Table 1 also shows exemplarily for test Nr. 1 (pure HPMC powder as the comparative test for the HPMC-batches) that the maximum viscosity (ηₘₐₓ) determined according to Protocol A is the same as that determined with Protocol B. In other words, both protocols are suited to determine maximum viscosity, with protocol B offering the benefit of also following the kinetics of the viscosity increase over time.

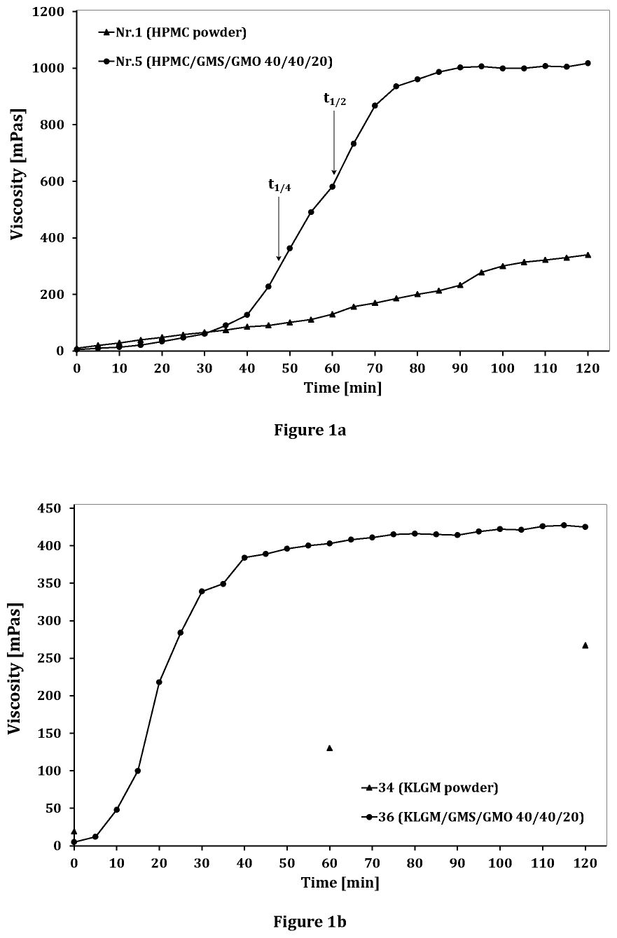

The exemplary viscosity kinetics of tests Nr. 1 (pure HPMC powder) and Nr. 5 (HPMC melt-granulated at lab-scale with a 2:1 GMS/GMO mixture), as well as tests Nr. 34 (pure KGLM powder) and Nr. 36 (KGLM melt-granulated at lab-scale with a 2:1 GMS/GMO mixture) are displayed in FIGS. 1a and 1b, respectively. As can be seen in FIGS. 1a and 1b, tests Nr. 5 and Nr. 36 exhibit sigmoidal curves with a lag-phase of delayed swelling (i.e. low viscosity levels, likely due to delayed disintegration of the lipid-containing compositions and thus delayed fibre dissolution), followed by a quick viscosity increase once progressive swelling occurs, and ultimately running into a plateau when approaching maximum viscosity (i.e. when most of the fibre is dissolved).

Regarding the seemingly-delayed viscosity increase of some of the comparative tests (for instance, pure HPMC powder in Table 1 or FIG. 1a), it should be noted that this is the outcome of some viscous fibre forming ‘gel lumps’ very quickly when attempting to disperse them in water or another aqueous medium. The outer gelled layer then prevents or delays the excess of water to the remaining powder ‘trapped’ inside the gel layer and thereby delays the fibre's dissolution and the resulting viscosity increase. This particular swelling behaviour is neither desirable in terms of administering the ‘lumpy’, poorly dispersible slurry to a subject, or patient, nor in terms of its effectiveness after ingestion.

### Example 6—Synergistic Effect of Single Lipids on HPMC Viscosity

Table 2 below shows the synergistic effects of melt-granulating (according to Ex. 1), or melt-extruding (according to Ex. 2), the viscous fibre hydroxypropylmethyl-cellulose (HPMC) with various single lipids; namely glycerol monostearate (GMS), glycerol monooleate (GMO), Witepsol® E85 (E85), and Sorbitan monooleate (Span 65), Witepsol® W25 (W25), glycerol monooleate (GMO), capric acid and myristic acid.

TABLE 2

| | Composition (prepared acc. to | | | ηₘₐₓ | t_{1/2} | t_{1/4} | t_{1/4}/t_{1/2} |
|---|---|---|---|---|---|---|---|
| Nr. | Ex. 1-3, tested acc. to Ex. 4A-C) | | | [mPas] | [min] | [min] | [%] |
| 1 | HPMC powder (100) | — | B | 920 | 180 | 90 | 50 |
| 7 | GMS/GMO lipid granules | 1 | A | 5 | — | — | — |
| | (66.7/33.3) | | | | | | |
| 2 | HPMC/GMS | 1 | B | 1081 | 59 | 47 | 80 |
| | (40/60) | | | | | | |
| 3 | HPMC/E85 | 1 | B | 1059 | 122 | 104 | 85 |
| | (40/60) | | | | | | |
| 22 | HPMC/Span 65 | 1 | B | 1007 | 160 | 111 | 69 |
| | (40/60) | | | | | | |
| 72 | HPMC/capric acid | 2 | B | 1069 | 61 | 53 | 87 |
| | (40/60) | | | | | | |
| 73 | HPMC/W25 | 2 | B | 1085 | 80 | 67 | 84 |
| | (40/60) | | | | | | |
| 80 | HPMC/myristic acid | 2 | B | 1213 | 86 | 75 | 87 |
| | (40/60) | | | | | | |

As seen in Example 6 and Table 2, melt-formulating HPMC with the above lipid components (e.g. as melt-granules or melt-extrudates according to Example 1 or 2, respectively) leads to a synergistic increase in maximum viscosity (ηₘₐₓ) in water at room temperature by at least 10% or more, and, beneficially, to faster swelling of the fibre, as indicated by the shorter half-times (t_{1/2}) in comparison to HPMC powder alone (Nr. 1). Presumably, the faster swelling up to maximum viscosity is due to the fact that no ‘gel lumps’ were formed during dispersion of the lipid-containing compositions and their disintegration in water. The synergistic increase in maximum viscosity, however, is surprising in so far as neither the HPMC alone, nor the lipids alone (as illustrated by the lipids mixture Nr. 7), nor the sum thereof can explain this increase in maximum viscosity.

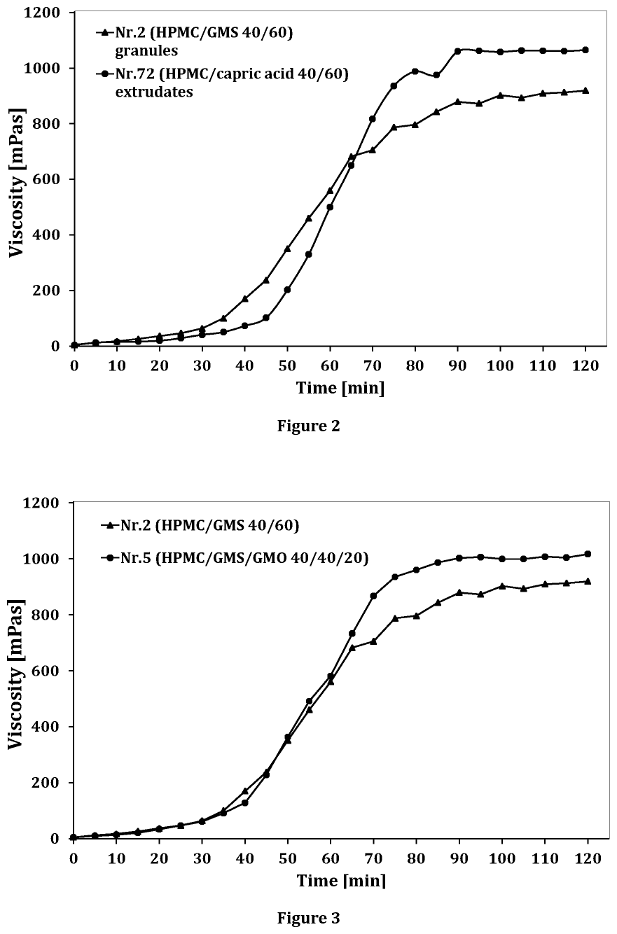

The exemplary viscosity kinetics of test Nr. 2 (HPMC melt-granulated at lab-scale with GMS) and Nr. 72 (HPMC melt-extruded at lab-scale with capric acid) are displayed in FIG. 2, and as can be seen, both tests Nr. 2 and 72 exhibit sigmoidal viscosity curves.

### Example 7—Synergistic Effect of Lipid Mixtures on HPMC Viscosity

Table 3 below shows the synergistic effects of melt-granulating (according to Ex. 1), or melt-extruding (according to Ex. 2 or 3), the viscous fibre hydroxypropylmethyl-cellulose (HPMC) with lipid mixtures; for instance, glycerol monostearate/glycerol monooleate (GMS/GMO), GMS/GMO/lecithin (GMS/GMO/LCT), tristearin/GMO (TS/GMO), TS/Tween, candelilla wax/GMO, GMS/glycerol, GMS/sorbitan monooleate (GMS/Span 80), or myristic acid/caprylic acid (myristic/caprylic).

TABLE 3

| | Composition (prepared acc. to | | | ηₘₐₓ | t_{1/2} | t_{1/4} | t_{1/4}/t_{1/2} |
|---|---|---|---|---|---|---|---|
| Nr. | Ex. 1-3, tested acc. to Ex. 4A-C) | | | [mPas] | [min] | [min] | [%] |
| 1 | HPMC powder (100) | — | B | 920 | 180 | 90 | 50 |
| 7 | GMS/GMO lipid granules | 1 | A | 5 | — | — | — |
| | (66.7/33.3) | | | | | | |
| 5 | HPMC/GMS/GMO | 1 | B | 1215 | 62 | 47 | 76 |
| | (40/40/20) | | | | | | |
| 6 | HPMC/GMS/GMO/LCT | 1 | B | 1320 | 85 | 71 | 84 |
| | (39/39/20/2) | | | | | | |
| 8 | HPMC/GMS/GMO | 2 | B | 1224 | 49 | 42 | 86 |
| | (40/40/20) | | | | | | |
| 9 | HPMC/GMS/GMO/LCT | 3 | B | 1449 | 69 | 63 | 91 |
| | (39/39/20/2) | | | | | | |
| 18 | HPMC/TS/GMO | 1 | B | 1004 | 67 | 52 | 78 |
| | (40/40/20) | | | | | | |
| 20 | HPMC/TS/Tween | 1 | B | 1094 | 26 | 10 | 38 |
| | (40/40/20) | | | | | | |
| 27 | HPMC/Candellila/GMO | 1 | B | 1015 | 46 | 35 | 76 |
| | (40/40/20) | | | | | | |
| 49 | HPMC/GMS/Glycerol | 1 | B | 1132 | 61 | 48 | 79 |
| | (40/40/20) | | | | | | |
| 50 | HPMC/GMS/Span 80 | 1 | B | 1030 | 45 | 32 | 71 |
| | (40/40/20) | | | | | | |
| 78 | HPMC/GMS/liquid GMO | 1 | B | 1110 | 34 | 24 | 71 |
| | (40/45/15) | | | | | | |
| 79 | HPMC/GMS/IWT372 | 1 | B | 1059 | 62 | 54 | 87 |
| | (40/55/5) | | | | | | |
| 87 | HPMC/myristic/caprylic | 2 | B | 1136 | 41 | 19 | 46 |
| | (40/55/5) | | | | | | |

As can be seen from Table 3, also combinations of lipids in form of molten mixtures (in which the HPMC fibre is embedded) are capable of achieving the synergistic increase of the maximum viscosity compared to the maximum viscosity achieved by the fibre alone or by the lipids; in particular, lipid mixtures combining higher-melting lipids (such as GMS/Mp˜71-72° C., TS/72-73° C., candelilla/Mp 69-70° C., or myristic acid/Mp˜53° C.) with lower-melting lipids (such as GMO/Mp 30° C., glycerol/Mp 18° C. caprylic acid/Mp 16° C., or Tween/Mp−21° C.).

For instance, 4 g of pure HPMC powder (Nr. 1) in 600 mL water yield a maximum viscosity (η_{max_fibre}) at room temperature of 920 mPas; 6 g of a 2:1 mixture of GMS/GMO (Nr. 7) under same conditions yield a maximum viscosity (η_{max_other}/η_{max_lipid}) of only 5 mPas; i.e. in sum 925 mPas. However, 10 g of the respective melt-granulated composition Nr. 5 (HPMC/GMS/GMO (40/40/20) yield a maximum viscosity (η_{max_syn}) of 1215 mPas in water at room temperature, or, in other words, a synergistic increase of over 30%. FIG. 3 shows the exemplary viscosity kinetics of test Nr. 2 (HPMC melt-granulated at lab-scale with GMS only) and Nr. 5 (HPMC melt-granulated at lab-scale with 2:1 GMS/GMO) in comparison. As can be seen, the addition of the lower-melting GMO (Mp 30° C.) to GMS (Mp 71-72° C.) did not change the viscosity kinetics in water at room temperature much but led to a further increase in the maximum viscosity (1081 mPas for GMS, 1215 mPas for GMS/GMO).

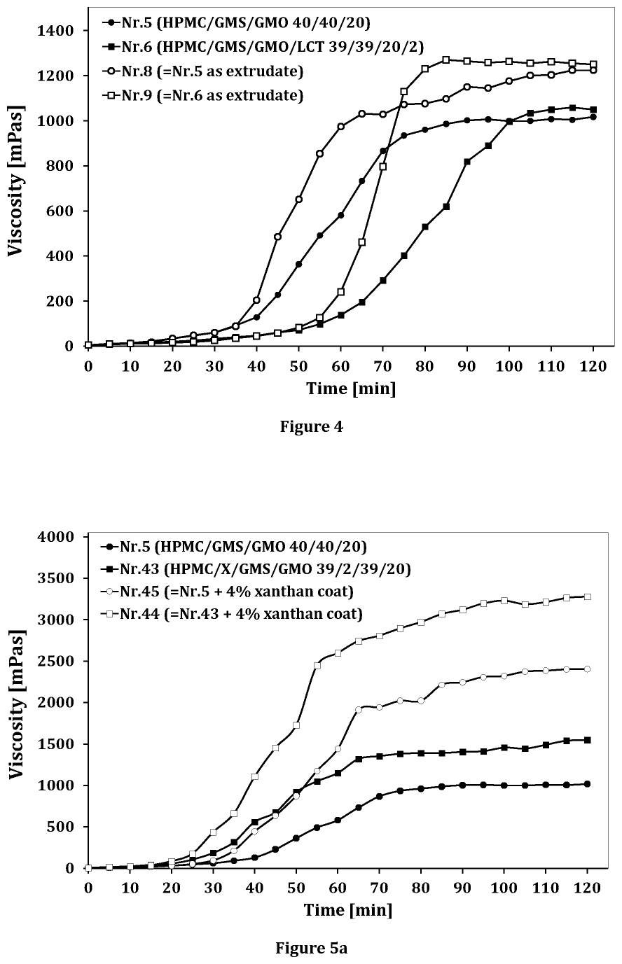

A further small increase was achieved by the addition of a small fraction of lecithin (about 3.3 wt.-% based on the weight of the lipids); see e.g. 1320 mPas achieved in water at room temperature by the melt-granules of test Nr. 6 (HPMC/GMS/GMO/LCT; 39/39/20/2) with lecithin, compared to 1215 mPas by the melt-granules of Nr. 5 without lecithin (HPMC/GMS/GMO; 40/40/20). The same increasing effect on the viscosity maximum by addition of lecithin was also observed with the respective melt-extrudates, namely, Nr. 8 (HPMC/GMS/GMO; 40/40/20) and Nr. 9 (HPMC/GMS/GMO/LCT; 39/39/20/2); yielding, in water at room temperature, 1449 mPas with lecithin compared to 1224 mPas without it. FIG. 4 shows the four tests in comparison. Interestingly, as can also be seen from FIG. 4 (e.g. comparing tests Nr. 5 with 6, and Nr. 8 with 9), the addition of lecithin furthermore resulted in a delay of about 15-20 min between the two viscosity kinetics curves. Hence, while depicted tests Nr. 5 and Nr. 8 showed suitable lag times of about 30-35 min even without lecithin, the addition of lecithin may be a valuable tool for other cases where the swelling of a composition occurs faster than intended (e.g. with a lag-time shorter than 15 min).

An even further, and more pronounced, increase of the maximum viscosity was possible by compacting the HPMC/GMS/GMO/LCT (39/39/20/2) composition further, for instance by melt-extruding it as exemplified in the production scale process of Example 3; see e.g. test Nr 9 which yielded a maximum viscosity (η_{max_syn}) of 1449 mPas in water at room temperature, compared to 1320 mPas by the lab-scale melt-granules of Nr 6. Similar results were found for tests Nr. 5 and 8, where the same HPMC/GMS/GMO (40/40/20) composition was prepared once as lab-scale melt-granules (Nr 5), and once as lab-scale melt-extrudates (Nr 8), with the extrudates of Nr. 8 yielding a slightly higher maximum viscosity (η_{max_syn}) of 1224 mPas in water at room temperature, compared to 1215 mPas by the lab-scale melt-granules of Nr 5. Additionally, and as shown in FIG. 4, test Nr. 5 compared to test Nr. 8, the use of compaction techniques, such as in an extruder, also appears to lead to faster disintegration of the particles of the synergistic fibre composition and quicker dissolution of the dietary fibre, as indicated by the shorter t_{1/2} and t_{1/4} times.

As mentioned above, and without wishing to be bound by theory, it is believed that the underlying mechanism of these synergistic increases of the maximum viscosity may involve the homogenous distribution of the dietary fibres, e.g. HPMC, in the lipids of the synergistic fibre compositions which promotes a far more homogenous dispersion of the viscous fibre in aqueous media such as water, and may thus facilitate a constant and fast hydration, and ultimately dissolution, of said fibre.

A further important characteristic for homogenous dispersion of the synergistic fibre compositions, and ultimately the fibre, in aqueous media is good wettability of the respective composition, and in particular of the lipid(s) under (b), which form most of the composition's outer surface. A surface that is easily hydrated is thus preferable. Without wishing to be bound by theory, it is assumed that, for instance, test Nr. 18 (HPMC/TS/GMO; 40/40/20) yields a lower maximum viscosity (η_{max_syn}) of 1004 mPas in water at room temperature, compared to 1215 mPas for the similar GMS-based granules of test Nr 5 (HPMC/GMS/GMO; 40/40/20), and also requires a bit more time for it (as indicated by Nr. 18's slightly longer t_{1/2} and t_{1/4} times), because tristearin (TS) is the more hydrophobic lipid compared to glycerol monostearate (GMS).

### Example 8—Effect of Xanthan on Synergistic Viscosity Increase and Viscosity Kinetics

In line with the above reasoning that the surface wettability of the synergistic fibre composition may play a role in achieving the synergistic increase in viscosity—preferably along with sigmoidal viscosity kinetics—further attempts were made in which the synergistic fibre compositions were modified with a compound providing good wettability; herein e.g. the dietary fibre xanthan. The xanthan, can either be embedded in the lipid(s) together with the dietary fibre, and/or it can be top-coated, or surface-coated, onto the synergistic fibre composition; for instance, as a xanthan powder-layer.

Table 4 below shows the synergistic effects of melt-granulating (according to Ex. 1), or melt-extruding (according to Ex. 2 or 3), the exemplary viscous fibres hydroxypropyl-methylcellulose (HPMC) and psyllium with various lipids or lipid mixtures, and adding xanthan to the compositions; for instance, a xanthan powder-layer at a coating level of 2 to 7 wt.-%, such as 3 wt.-%, 4 wt.-%, based on the weight of the uncoated cores. Alternatively, the xanthan may be embedded together with the fibre within the lipid(s) so as to be part of the core. The numbers in bracket indicate the weight ratios within the core, and—where present—the “+ . . . X”-provision at the end, such as “+4X”, indicates the coating level (here 4 wt.-%) for the compositions where the xanthan was applied as a top-coating. Optionally, xanthan can be both, embedded within the lipid as part of the core, and top-coated onto the same core; see e.g. test Nr. 15 or Nr. 64.

TABLE 4

| | Composition (prepared acc. to | | | ηₘₐₓ | t_{1/2} | t_{1/4} | t_{1/4}/t_{1/2} |
|---|---|---|---|---|---|---|---|
| Nr. | Ex. 1-3, tested acc. to Ex. 4A-C) | | | [mPas] | [min] | [min] | [%] |
| 12a | HPMC/xanthan powder mix | — | A | 1497 | — | — | — |
| | (91:9) | | | | | | |
| 12b | HPMC/xanthan powder mix | — | A | 2864 | — | — | — |
| | (85:15) | | | | | | |
| 7 | GMS/GMO lipid granules | 1 | A | 5 | — | — | — |
| | (66.7/33.3) | | | | | | |
| 3 | HPMC/E85 | 1 | | 1059 | 122 | 104 | 85 |
| | (40/60) | | | | | | |
| 88 | HPMC/E85 + | 1 | B | 1670 | 101 | 86 | 85 |
| | 4% xanthan powder-layer | | | | | | |
| | (40/60/+4X) | | | | | | |
| | HPMC/GMS/GMO | 1 | B | 1215 | 62 | 47 | 76 |
| | (40/40/20) | | | | | | |
| 4.5 | HPMC/GMS/GMO + | 1 | B | 2551 | 57 | 45 | 79 |
| | 4% xanthan powder-layer | | | | | | |
| | (40/40/20/+4X) | | | | | | |
| 4.3 | HPMC/X//GMS/GMO | 1 | B | 1590 | 47 | 36 | 77 |
| | (39/2/39/20) | | | | | | |
| 44 | HPMC/X/GMS/GMO + | | B | 3300 | 49 | 37 | 76 |
| | 4% xanthan powder-layer | | | | | | |
| | (39/2/39/20/+4X) | | | | | | |
| 6 | HPMC/GMS/GMO/LCT | 1 | B | 1320 | 86 | 72 | 84 |
| | (39/39/20/2) | | | | | | |
| 10 | HPMC/GMS/GMO/LCT + | 1 | B | 2513 | 57 | 49 | 86 |
| | 4% xanthan powder-layer | | | | | | |
| | (39/39/20/2/+4X) | | | | | | |
| 14 | HPMC/X/GMS/GMO/LCT | 1 | B | 1648 | 46 | 35 | 76 |
| | (38.5/2/38.5/19/2) | | | | | | |
| 15 | HPMC/X/GMS/GMO/LCT + | 1 | B | 3382 | 48 | 38 | 79 |
| | 4% xanthan powder-layer | | | | | | |
| | (38.5/2/38.5/19/2/+4X) | | | | | | |
| 9 | HPMC/GMS/GMO/LCT | 3 | B | 1449 | 69 | 63 | 91 |
| | (39/39/20/2) | | | | | | |
| 11 | HPMC/GMS/GMO/LCT + | 3 | B | 4181 | 65 | 58 | 89 |
| | 4% xanthan powder-layer | | | | | | |
| | (39/39/20/2/+4X) | | | | | | |
| 18 | HPMC/TS/GMO | 1 | B | 1004 | 67 | 52 | 78 |
| | (40/40/20) | | | | | | |
| 19 | HPMC/TS/GMO + | 1 | B | 1980 | 63 | 47 | 75 |
| | 3% xanthan powder-layer | | | | | | |
| | (40/40/20/+3X) | | | | | | |
| 20 | HPMC/TS/Tween | 1 | B | 1094 | 26 | 10 | 38 |
| | (40/40/20) | | | | | | |
| 21 | HPMC/TS/Tween + | 1 | B | 4371 | 23 | 17 | 74 |
| | 7% xanthan powder-layer | | | | | | |
| | (40/40/20/+7X) | | | | | | |
| 27 | HPMC/Candellila/GMO | 1 | B | 1015 | 46 | 35 | 76 |
| | (40/40/20) | | | | | | |
| 28 | HPMC/Candellila/GMO + | 1 | B | 2033 | 50 | 34 | 68 |
| | 4% xanthan powder-layer | | | | | | |
| | (40/40/20/+4X) | | | | | | |
| 52 | HPMC/GMS/Glycerol | 1 | B | 1024 | 59 | 46 | 78 |
| | (40/55/5) | | | | | | |
| 53 | HPMC/GMS/Glycerol + | 1 | B | 2126 | 69 | 52 | 75 |
| | 4% xanthan powder-layer | | | | | | |
| | (40/55/5/+4X) | | | | | | |
| 8 | HPMC/GMS/GMO | 2 | B | 1224 | 49 | 42 | 86 |
| | (40/40/20) | | | | | | |
| 61 | HPMC/X/GMS/GMO | 2 | B | 2907 | 56 | 47 | 84 |
| | (38.5/4/38.5/19) | | | | | | |
| 62 | HPMC/X/GMS/GMO + | 2 | B | 5376 | 51 | 43 | 84 |
| | 4% xanthan powder-layer | | | | | | |
| | (38.5/4/38.5/19/+4X) | | | | | | |
| 64 | HPMC/X/GMS/liquid GMO +9 | 2 | B | 2197 | 49 | 41 | 84 |
| | 2% xanthan powder-layer | | | | | | |
| | (39/2/54/5/+2X) | | | | | | |
| 91 | HPMC/GMS/liquid GMO + | 2 | B | 2717 | 51 | 42 | 82 |
| | 4% xanthan powder-layer | | | | | | |
| | (40/55/5/+4X) | | | | | | |
| 30 | Psyllium/xanthan powder | — | A | 244 | — | — | — |
| | (91:9) | | | | | | |
| 32 | Psyllium/GMS/GMO | 2 | B | 371 | 68 | 48 | 71 |
| | (40/40/20) | | | | | | |
| 33 | Psyllium/GMS/GMO + | 2 | B | 471 | 50 | 30 | 60 |
| | 4% xanthan powder-layer | | | | | | |
| | (40/40/20/+4X) | | | | | | |

For both viscous fibres, HPMC and psyllium, tested in water at room temperature, it is shown in Table 4, that the provision of a xanthan powder-layer as a top-coating provides an increase in viscosity of at least 50% over the compositions without such top-coat, typically more than 50%. For instance, the Witepsol® JE85-based HPMC melt-granules of test Nr. 88 yield a ca. 58% higher viscosity maximum than those of related test Nr.3 without the xanthan powder-layer. The HPMC-granules of test Nr. 45, formulated with a 2:1 GMS/GMO mixture, yield a ca. 110% higher viscosity maximum than related Nr. 5 without the xanthan powder-layer. The HPMC-granules of test Nr. 10, formulated with a 2:1 GMS/GMO/lecithin mixture (said GMS/GMO/LCT-mixture exbiting a ‘joint’ Mp of 65.3° C.), yield a ca. 90% higher viscosity maximum than related Nr. 6 without the xanthan powder-layer. The production scale HPMC-extrudates of test Nr. 11, based on the same GMS/GMO/LCT-mixture, even yield a ca. 189% higher viscosity maximum than related Nr. 9 without the xanthan powder-layer.

Tests Nr. 5 and 43 as well as tests Nr. 6 and 14, show the effect of embedding a small amounts of xanthan (here 5 wt.-% based on the weight of the dietary fibre, or in other words at fibre-to-xanthan ratio of ca. 95:5) directly into the core, together with the dietary fibre. Tests Nr. 8 and 61 show the same for a 10 wt.-% xanthan addition (fibre-to-xanthan ratio 91:9). All yield an increase of the maximum viscosity of at least 20% in water at room temperature over the compositions without such xanthan addition to the core; for instance, ca. 31% for Nr. 43 compared to Nr. 5, ca. 25% for Nr. 14 compared to Nr. 6, and ca. 138% for Nr. 61 compared to Nr. 8. Furthermore, the addition of these small amounts of xanthan (e.g. adding 0.2 g or 0.4 g xanthan to 4 g HPMC) can, in some cases, also accelerate disintegration in aqueous media such as water, as indicated by the shorter t_{1/2} and t_{1/4} times. This finding also applies when an additional xanthan powder-layer is added to the same compositions, as seen for compositions Nr. 10 and Nr. 15.

Test Nr. 15 is an example of a lab-scale granule composition (prepared according to Example 1) where xanthan is both embedded within the lipid as part of the core (see Nr. 14) and top-coated onto the same core. The HPMC-granules of test Nr. 15 yield a ca. 105% higher viscosity maximum in water at room temperature than the related Nr. 14 without the xanthan powder-layer. Compared to the related test Nr. 6 which is free of xanthan (i.e. neither in the core, nor as a coating), test Nr. 15 even yields a ca. 156% higher viscosity maximum in water at room temperature. A similar trend can be seen with the xanthan-top-coated HPMC-granules of tests Nr. 44 compared to Nr. 43 and Nr. 5, as well as with the HPMC-extrudates of tests Nr. 62 compared to Nr. 61 and Nr. 8.

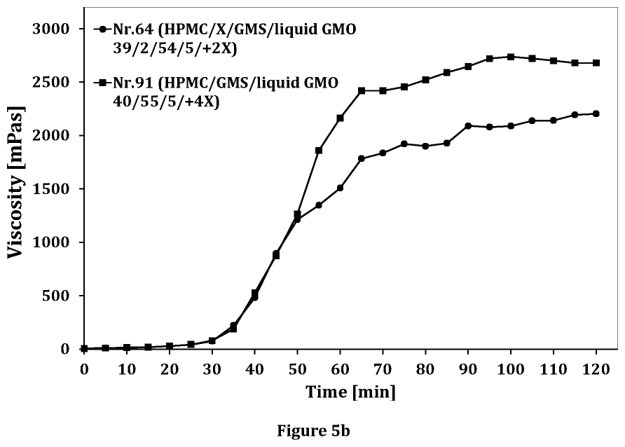

The above-described effects of xanthan additions to a melt-granulated core and/or to its surface (in form of a powder-layer top coating) are depicted exemplarily for test Nr 5 and 43 to 45 in FIG. 5a.

FIG. 5b shows tests Nr. 64 and Nr. 91 which are examples of lab-scale HPMC-extrudates (prepared according to Example 2) where 0.4 g xanthan is added to 4 g HPMC; in case of Test Nr. 91 all of the xanthan is top-coated, and in case of Nr. 64 it is split evenly between being embedded within the lipid as part of the extruded core (i.e. 0.2 g xanthan inside the core together with 4 g HPMC) and top-coated onto the core (further 0.2 g xanthan). As can be seen form Table 4 and FIG. 5b, when applied as a top-coating, the added xanthan is more effective in increasing the maximum viscosity.

The effect of xanthan on psyllium as the dietary fibre is slightly less pronounced than with HPMC. The psyllium-extrudates of test Nr. 33, formulated with a 2:1 GMS/GMO mixture, yield a ca. 27% higher viscosity maximum in water at room temperature than related Nr. 32 without the xanthan powder-layer.

Furthermore, all xanthan-containing compositions of Table 4 exhibit a synergistic increase of the maximum viscosity of said composition in water at room temperature compared to the maximum viscosity achieved by the fibre and the lipids together. For instance, the GMS/GMO-based HPMC-granules of test Nr. 10 yield a ca. 67% higher viscosity maximum in water at room temperature than the HPMC/xanthan powder blend (91:9) of test Nr. 12a and the GMS/GMO lipid granules of test Nr. 7 together (1497+5 mPas). In other words, xanthan can be used to enhance the synergistic effect on the synergistic fibre composition's maximum viscosity according to the present invention, namely the effect achieved embedding the dietary fibre(s) into lipid(s).

Despite the increases of the viscosity maxima of the xanthan-containing synergistic fibre compositions in water at room temperature, the time periods for disintegration of the compositions, as well as the progression of viscosity upon dissolution of the fibre, is nearly unchanged. Rather on the contrary, Table 4 shows that for the majority of tested compositions, the incorporation of xanthan even shortens the times t_{1/4} and t_{1/2} until reaching the above-mentioned increased viscosity maxima.

### Example 9—Effect of Temperature and Aqueous Medium on Synergistic Viscosity Increase and Viscosity Kinetics

The tests described above were mostly performed water at room temperature (see Example 4, Protocols A and B above). To mimic the digestive process of the compositions, further tests were performed on the viscosity maxima and kinetics in fasted state simulated gastric fluid (FaSSGF) at constant temperatures of 37° C. As shown in Table 5, the maximum viscosities of dietary fibres such as HPMC and psyllium are generally lower in simulated gastric fluid at 37° C. (Protocol C) compared to water at room temperature (Protocol A or B), which is expected due to the higher temperature. However, the findings of a synergistic effect by embedding these fibres into lipids on the maximum viscosities, the shorter t_{1/2} and t_{1/4} values (i.e. faster swelling to said maximum), as well as the beneficial impact of xanthan addition are still present at 37° C. and in FaSSGF. For instance, test Nr. 11 still yields a >90% higher viscosity maximum in FaSSGF than related Nr. 12a in the same medium and temperature.

TABLE 5

| | Composition (prepared acc. to | | | ηₘₐₓ | t_{1/2} | t_{1/4} | t_{1/4}/t_{1/2} |
|---|---|---|---|---|---|---|---|
| Nr. | Ex. 1-3, tested acc. to Ex. 4A-C) | | | [mPas] | [min] | [min] | [%] |
| 1 | HPMC powder (100) | — | B | 920 | 180 | 90 | 50 |
| | HPMC powder (100) | — | C | 436 | 240 | 120 | 50 |
| 12a | HPMC/xanthan powder mix | — | A | 1497 | — | — | — |
| | (91:9) | | | | | | |
| 12a | HPMC/xanthan powder mix | — | C | 893 | 150 | 95 | 63 |
| | (91:9) | | | | | | |
| 29 | Psyllium powder (100) | — | A | 84 | — | — | — |
| 46 | Psyllium powder (100) | — | C | 24 | 20 | 15 | 75 |
| 11 | HPMC/GMS/GMO/LCT + | 3 | E | 4181 | 65 | 58 | 89 |
| | 4% xanthan powder-layer | | | | | | |
| | (39/39/20/2/+4X) | | | | | | |
| 11 | HPMC/GMS/GMO/LCT + | 3 | C | 1730 | 68 | 61 | 90 |
| | 4% xanthan powder-layer | | | | | | |
| | (39/39/20/2/+4X) | | | | | | |
| 61 | HPMC/X/GMS/GMO | 2 | B | 2907 | 56 | 47 | 84 |
| | (38.5/4/38.5/19) | | | | | | |
| 61 | HPMC/X/GMS/GMO | 2 | C | 1014 | 62 | 53 | 85 |
| | (38.5/4/38.5/19) | | | | | | |

### Example 10—Effect of Embedding the Dietary Fibre(s) in the Lipids on the Synergistic Viscosity Increase in FaSSGF at 37° C.

In order to show that the synergistic enhancement of the viscosity maximum results from the specific formulation of viscous dietary fibre(s) with the lipids, specifically from embedding them therein, a comparative test for the extrudates of Nr. 11 was run in fasted state simulated gastric fluid (FaSSGF) at 37° C.; see Table 6.

TABLE 6

| | Composition (prepared acc. to | | | ηₘₐₓ | t_{1/2} | t_{1/4} | t_{1/4}/t_{1/2} |
|---|---|---|---|---|---|---|---|
| Nr. | Ex. 1-3, tested acc. to Ex. 4A-C) | | | [mPas] | [min] | [min] | [%] |
| 1 | HPMC powder (4 g) | — | C | 436 | 240 | 120 | 50 |
| 12a | HPMC/xanthan powder mix | — | C | 893 | 150 | 95 | 63 |
| | (4 g + 0.4 g) | | | | | | |
| 7 | GMS/GMO | 1 | C | <5 | — | — | — |
| | (4 g + 2 g) | | | | | | |
| 16 | HPMC/X/LCT powder mix | 1 | C | 672 | — | — | — |
| | (4 g + 0.4 g + 0.2 g) + | | | | | | |
| | GMS/GMO lipid granule of No. 7 | | | | | | |
| | (+4 g + 2 g) | | | | | | |
| 11 | HPMC/GMS/GMO/LCT | 3 | C | 1730 | 68 | 61 | 90 |
| | (4 g + 4 g + 2 g + 0.2 g) + | | | | | | |
| | 0.4 g xanthan powder-layer | | | | | | |

As can be seen in Table 6, when an amount of 6 g of the 2:1 GMS/GMO-granules of Nr. 7 (i.e. 4 g GMS and 2 g GMO) are tested together with a mere dry powder mix of 4 g HPMC, 0.4 g xanthan and 0.2 g lecithin, the maximum viscosity achieved in FaSSGF at 37° C. is only 672 mPas (test Nr. 16). However, when the same amounts are formulated according to the present invention, namely, when the 4 g HPMC are embedded within a molten mixture of 4 g GMS, 2 g GMO, and 0.2 g lecithin by extrusion as described in Example 3, with the 0.4 g xanthan applied as a powder top-coating layer thereto (test Nr. 11), then the maximum viscosity achieved in FaSSGF at 37° C. is increased quite significantly to 1730 mPas. In other words, the mere addition of lipids to the dietary fibre(s) does not exhibit the same synergistic effect as embedding the fibre(s) in the lipids, thereby emphasizing the unique effect of viscosity enhancement of the present invention.

This finding is particularly surprising in so far that, in absence of GMS, GMO and lecitihin, a pure powder blend of 4 g HPMC and 0.4 g xanthan (test Nr. 12a) achieves a maximum viscosity in FaSSGF at 37° C. of 893 mPas, and thus yielding a higher maximum viscosity than the 672 mPas achieved by test Nr. 16. It is almost as if the mere addition of the lipids to the dietary fibres in form can, in some cases, lower the maximum viscosity, while a more homogenous formulation of these components—mainly the embedding of the dietary fibre(s) within the lipid material (and, optionally, embedding or top-coating xanthan if present)—achieves a synergistic increase of the maximum viscosity.

The following list of numbered items are embodiments comprised by the present invention:
1. A synergistic fibre composition comprising:
   (a) a dietary fibre selected from the group consisting of hydroxpropylmethylcellulose (HPMC), methylcellulose (MC), carboxymethylcellulose (CMC), psyllium seed husk, xanthan gum, galactomannan (e.g. from guar gum or locust bean gum), and glucomannan (e.g. from konjac gum); and
   (b) a lipid material comprising a lipid selected from:
      (b₁) a free fatty acid exhibiting a melting point in the range of from 30 to 60° C.,
      (b₂) a glycerol- or sorbitan based fatty acid ester exhibiting a melting point in the range of from 30 to 75° C.,
      (b₃) a plant-derived wax exhibiting a melting point in the range of from 30 to 75° C., or
      a homogenous mixture of two or more of the lipids under bi, b₂ and/or b₃,
      wherein the homogenous mixture of these lipids exhibits a melting point in the range of from 30 to 70° C.,
      wherein the lipid material under (b) comprises at least one lipid under b₁ to b₃ which is selected from the group consisting of myristic acid, capric acid,
      glycerolmonostearate (GMS), tristearin, and candelilla wax;
   wherein the dietary fibre is embedded within the lipid material; and
   wherein the synergistic fibre composition is capable of modifying the swelling behavior of the dietary fibre in such a way that:
      the maximum viscosity that is achieved by mixing the synergistic fibre composition, comprising the dietary fibre(s) at an amount x_{_fibre}, in a given volume Vₓ of the aqueous medium (η_{max_syn}) is higher than the maximum viscosity achieved in the same volume Vₓ of the aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}), and higher than the cumulative maximum viscosity of η_{max_fibre} plus η_{max_other} (η_{max_Σ}); and optionally
      the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.
2. The composition according to item 1, wherein the weight ratio of the dietary fibre under (a) to the lipid material under (b) in the synergistic fibre composition is in the range from 30:70 to 60:40, or from 33:67 to 55:45, or from 35:65 to 50:50, or from 35:65 to 45:55, or from 37:63 to 42:58.
3. The composition according to any one of items 1 or 2, wherein the synergistic fibre composition comprises at least 45 wt.-%, or at least 50 wt.-%, or at least 55 wt.-%, or at least 60 wt.-% of the lipid material under (b) based on the total weight of the synergistic fibre composition.
4. The composition according to any one of items 1 to 3, wherein, the synergistic fibre composition comprises at least 50 wt.-% of the lipid material under (b) based on the total weight of the synergistic fibre composition; and the weight ratio of the dietary fibre under (a) to the lipid material under (b) in the synergistic fibre composition is in the range from 35:65 to 50:50.
5. The composition according to any one of items 1 to 4, wherein the dietary fibre under (a) is melt-embedded within, optionally melt-extruded with, the lipid material under (b).
6. The composition according to any one of items 1 to 5, wherein the synergistic fibre composition does not comprise any dietary fibres other than a dietary fibre selected from the group consisting of hydroxypropylmethylcellulose (HPMC), methylcellulose (MC), carboxymethylcellulose (CMC), psyllium seed husk, xanthan gum, galactomannan, and glucomannan.
7. The composition according to any one of items 1 to 6, wherein the synergistic fibre composition is substantially free of poly(carboxylates), poly(methacrylic acid), copolymers of acrylic and methacrylic acid, poly(hydroxyethyl methacrylic acid), alginic acid or salts thereof, microcrystalline cellulose, chitosan, gum arabic, beta glucan and pectins.
8. The composition according to any one of items 1 to 7, wherein the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, of a lipid selected from the group consisting of monoglycerides, triglycerides, and sorbitan-fatty acid triesters, based on the total weight of the lipid material (b).
9. The composition according to any one of items 1 to 8, wherein the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b).
10. The composition according to any one of items 1 to 9, wherein the lipid material under (b) further comprises an additional lipid (b) in a homogenous mixture with the one or more lipid(s) under bi, b₂ and/or b₃, wherein additional lipid (b′) is selected from the group consisting of:
   glycerol,
   a phosphatidylcholine,
   a sorbitan mono fatty acid ester,
   one or more fatty acid ester(s) selected from the group consisting of fatty acid esters of acetic acid, lactic acid, citric acid, tartaric acid, monoacetyl tartaric acid and diacetyl tartaric acid,
   one or more fatty acids with a melting point below 30° C.; and
   wherein the homogenous mixture exhibits a melting point in the range of from 30 to 70° C.
11. The composition according to any one of items 1 to 10, wherein the lipid material under (b) further comprises an additional lipid (b′) in a homogenous mixture with the one or more lipid(s) under b₁, b₂ and/or b₃, wherein the additional lipid (b′) is a phosphatidylcholine, optionally a lecithin such as egg lecithin, soy lecithin, or sunflower lecithin.
12. The composition according to any one of items 1 to 11, wherein the lipid material under (b) further comprises an additional lipid (b′) in a homogenous mixture with the one or more lipid(s) under b₁, b₂ and/or b₃, wherein the additional lipid (b′) is a sorbitan mono fatty acid ester, optionally a sorbitan mono fatty acid ester selected from the group consisting of sorbitanmonooleate, polyoxyethylen sorbitanmonolaurate, polyoxyethylen sorbitanmonostearate, and polyoxyethylen sorbitanmonooleate.
13. The composition according to any one of items 1 to 12, wherein the lipid material under (b₁) is selected from the group consisting of saturated fatty acids, C₈-C₁₄ fatty acids, and saturated C₈-C₁₄ fatty acids.
14. The composition according to any one of items 1 to 13, wherein the lipid material under (b₁) is selected from the group consisting of myristic acid and capric acid.
15. The composition according to any one of items 1 to 14, wherein the lipid material under (b₃) is a plant-derived wax selected from the group consisting of candelilla wax, carnauba wax, berry wax, myrica fruit wax, soy wax, rice bran wax, and bees wax.
16. The composition according to any one of items 1 to 15, wherein the lipid material under (b) comprises at least 70 wt.-%, or at least 75 wt.-%, or at least 80 wt.-%, or at least 85 wt.-%, or at least 90 wt.-%, of the lipids (b₁) to (b₃) based on the total weight of the lipid material (b).
17. The composition according to any one of items 1 to 16, wherein the lipid material under (b) consists of the lipids (b₁) to (b₃), or
   optionally of the lipids (b₁) to (b₃) and the additional lipids (b′).
18. The composition according to any one of items 1 to 17, wherein the lipid material under (b) exhibits a melting point in the range of from 30 to 70° C.
19. The composition according to any one of items 1 to 18, wherein the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b); and wherein the lipid material under (b) comprises at least one other lipid selected from the group consisting of caprylic acid, glycerol, and glycerolmonooleate (GMO).
20. The composition according to any one of items 1 to 19, wherein the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b); and wherein the lipid material under (b) comprises at least one other lipid selected from the group consisting of caprylic acid, glycerol, and glycerolmonooleate (GMO); and wherein the lipid material under (b) further comprises lecithin.
21. The composition according to any one of items 1 to 20, the synergistic fibre composition further comprises xanthan, either in the form of a powder top-coating layer, and/or with the xanthan being embedded in the lipid(s) under (b) together with the dietary fibre under (a); optionally, in a weight ratio of fibre to xanthan in the range of 98:2 to 75:25, 96:4 to 80:20, or 94:6 to 82:18; such as 91:9 or 85:15.
22. The composition according to any one of items 1 to 21, wherein the synergistic fibre composition further comprises xanthan in the form of a powder top-coating layer; and wherein the coating level thereof is in the range of 1 to 20 wt.-%, or 1 to 10 wt.-%, or 2 to 8 wt.-%, based on the total weight of the uncoated cores.
23. The composition according to any one of items 1 to 22, wherein the synergistic fibre composition comprises, or consists of, hydroxpropylmethylcellulose (HPMC) as the dietary fibre under (a); and glycerolmonostearate (GMS), glycerolmonooleate (GMO), and lecithin as the components of the lipid material under (b).
24. The composition according to any one of items 1 to 23, wherein the synergistic fibre composition comprises, or consists of, hydroxypropylmethylcellulose (HPMC) and xanthan as the dietary fibres under (a); and glycerolmonostearate (GMS), glycerolmonooleate (GMO), and lecithin as the components of the lipid material under (b).
25. The composition according to any one of items 1 to 24, wherein the synergistic fibre composition comprises, or consists of, hydroxypropylmethylcellulose (HPMC) and xanthan as the dietary fibres under (a); glycerolmonostearate (GMS), glycerolmonooleate (GMO), and lecithin as the components of the lipid material under (b);
   wherein the xanthan is provided either in the form of a powder top-coating layer on the synergistic fibre composition, and/or wherein the xanthan is embedded within the lipid material together with the hydroxypropylmethylcellulose (HPMC).
26. The composition according to any one of items 1 to 25, wherein the synergistic fibre composition comprises, or consists of, hydroxypropylmethylcellulose (HPMC) and xanthan as the dietary fibres under (a); glycerolmonostearate (GMS), glycerolmonooleate (GMO), and lecithin as the components of the lipid material under (b); and
   wherein the xanthan is provided in the form of a powder top-coating layer on the synergistic fibre composition.
27. The composition according to any one of items 1 to 26, wherein, based on its total weight, the synergistic fibre composition comprises, or consists of:
   from 25 to 55 wt.-%, or from 35 to 45 wt.-%,
   hydroxypropylmethylcellulose (HPMC),
   from 25 to 55 wt.-%, or from 35 to 45 wt.-%, glycerolmonostearate (GMS),
   from 10 to 30 wt.-%, or from 15 to 25 wt.-%, glycerolmonooleate (GMO),
   from 1 to 20 wt.-%, or from 1 to 10 wt.-%, lecithin, and
   from 1 to 20 wt.-%, or from 1 to 10 wt.-%, xanthan.
28. The composition according to any one of items 1 to 27, wherein, based on its total weight, the synergistic fibre composition comprises, or consists of:
   from 36 to 40 wt.-% hydroxypropylmethylcellulose (HPMC),
   from 36 to 40 wt.-% glycerolmonostearate (GMS),
   from 17 to 21 wt.-%, glycerolmonooleate (GMO),
   from 1 to 3 wt.-%, lecithin, and
   from 1 to 5 wt.-% xanthan.
29. The composition according to any one of items 1 to 28, wherein, based on its total weight, the synergistic fibre composition comprises, or consists of:
   from 36 to 40 wt.-% hydroxypropylmethylcellulose (HPMC),
   from 36 to 40 wt.-% glycerolmonostearate (GMS),
   from 17 to 21 wt.-%, glycerolmonooleate (GMO),
   from 1 to 3 wt.-%, lecithin, and
   from 1 to 5 wt.-% xanthan; and
   wherein the and the xanthan is provided either in the form of a powder top-coating layer on the synergistic fibre composition and/or embedded in the lipids together with the HPMC.
30. The composition according to any one of items 1 to 29, wherein the synergistic fibre composition is provided in the form of a plurality of dry, flowable, ingestible, particles, for instance as minitablets, granules, pellets, or powders, said ingestible particles optionally exhibiting a sieve diameter of above 0.01 mm, or above 0.05 mm, or above 0.1 mm, or above 0.2 mm, or above 0.3 mm; and/or
   a sieve diameter of up to 3.0 mm, or up to 2.0 mm, or up to 1.5 mm, or up to 1.0 mm, or up to 0.8 mm, or up to 0.6 mm, or up to 0.5 mm,
   such as a sieve diameter in the range of 0.01 mm-3.0 mm, or 0.1 mm-3.0 mm, or 0.2 mm-3.0 mm, or 0.5 mm-3.0 mm, or 0.1 mm-2.5 mm, or 0.2 mm-2.5 mm, or 0.5 mm-2.5 mm, or 0.1 mm-2.0 mm, or 0.2 mm-2.0 mm, or 0.5 mm-2.0 mm, or 0.1 mm-1.5 mm, or 0.2 mm-1.5 mm, or 0.5 mm-1.5 mm, or 0.1 mm-1.25 mm, or 0.2 mm-1.25 mm, or 0.5 mm-1.25 mm, or 0.1 mm-1.0 mm, or
   0.2 mm-1.0 mm, or 0.5 mm-1.0 mm, or 0.1 mm-0.8 mm, or 0.2 mm-0.8 mm, 0.5 mm-0.8 mm.
31. The composition according to any one of items 1 to 30, wherein the synergistic fibre composition is provided in the form of a plurality of dry, flowable, ingestible particles, for instance as minitablets, granules, pellets, or powders, and said ingestible particles are free of a synthetic drug substance.
32. The composition according to any one of items 1 to 31, wherein the viscosity in the aqueous medium is measured at a temperature in the range of from 15 to 40° C.
33. The composition according to any one of items 1 to 32, wherein the viscosity in the aqueous medium is measured at room temperature, or at a temperature of about 37° C.
34. The composition according to any one of items 1 to 33, wherein the viscosity in the aqueous is measured at room temperature, or at a temperature of about 37° C., and wherein the aqueous medium is selected from water, fasted-state simulated intestinal fluid (FaSSIF), fed-state simulated intestinal fluid (FeSSIF), fasted-state simulated intestinal fluid (FaSSGF), 0.1 N HCl, pH 6.8 phosphate-buffer solution, or similar bio-relevant media.
35. The composition according to any one of items 1 to 34, wherein, in water at room temperature, η_{max_syn} (the maximum viscosity that is achieved by mixing the synergistic fibre composition, comprising the dietary fibre(s) at an amount x_{_fibre}, in a given volume Vₓ of the water) is at least 10%, preferably at least 15%, more preferably at least 20%, higher than η_{max_Σ} (the cumulative maximum viscosity of η_{max_fibre} plus η_{max_other}).
36. The composition according to any one of items 1 to 35, wherein the synergistic fibre composition further comprises xanthan; and
   wherein, in water at room temperature, η_{max_syn} is at least 30%, preferably at least 40%, more preferably at least 50%, higher than η_{max_Σ}.
37. The composition according to any one of items 1 to 36, wherein the synergistic fibre composition further comprises xanthan in the form of a powder top-coating layer, at a coating level in the range of 1 to 20 wt.-%, or 1 to 10 wt.-%, or 2 to 8 wt.-%, based on the total weight of the uncoated cores; and
   wherein, in water at room temperature, η_{max_syn} in is at least 30%, preferably at least 40%, more preferably at least 50% higher than η_{max_Σ.}
38. The composition according to any one of items 1 to 37, wherein the swelling exhibits sigmoidal viscosity kinetics characterized by a lag time of at least 20 min, followed by a viscosity increase to at least 90% of the maximum viscosity (η_{max_syn}), that is achieved by mixing the synergistic fibre composition, comprising the dietary fibre(s) at an amount x_{_fibre}, in a given volume Vₓ of the aqueous medium, within no more than 2 h.
39. The composition according to any one of items 1 to 38, wherein the swelling behavior is modified in such a way that in water at room temperature:
   the maximum viscosity that is achieved by mixing the synergistic fibre composition, comprising the dietary fibre(s) at an amount x_{_fibre}, in a given volume Vₓ of the water (η_{max_syn}) is at least 10%, preferably at least 15%, more preferably at least 20% higher than the cumulative maximum viscosity of η_{max_fibre} plus η_{max_other} (η_{max_Σ}); and
   the swelling exhibits sigmoidal viscosity kinetics characterized by a lag time of at least 20 min, followed by a viscosity increase to at least 90% of the maximum viscosity (η_{max_syn}), that is achieved by mixing the synergistic fibre composition, comprising the dietary fibre(s) at an amount x_{_fibre}, in a given volume Vₓ of the water, within no more than 2 h.
40. A method of modifying the swelling behavior of a dietary fibre in an aqueous medium by providing a synergistic fibre composition comprising:
   (a) the dietary fibre, and
   (b) a lipid material comprising a lipid selected from:
      (b₁) a free fatty acid exhibiting a melting point in the range of from 30 to 60° C.,
      (b₂) a glycerol- or sorbitan based fatty acid ester exhibiting a melting point in the range of from 30 to 75° C.,
      (b₃) a plant-derived wax exhibiting a melting point in the range of from 30 to 75° C., or
      a homogenous mixture of two or more of the lipids under b₁, b₂ and/or b₃,
      wherein the homogenous mixture of these lipids exhibits a melting point in the range of from 30 to 70° C.;
   wherein the dietary fibre is embedded within the lipid material, and
   wherein the swelling behavior is modified in such a way that:
      the maximum viscosity that is achieved by mixing the synergistic fibre composition, comprising the dietary fibre(s) at an amount x_{_fibre}, in a given volume Vₓ of the aqueous medium (η_{max_syn}) is higher than the maximum viscosity achieved in the same volume Vₓ of the aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}), and higher than η_{max_Σ} (the cumulative maximum viscosity of η_{max_fibre} plus η_{max_other}); and optionally
      the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.
41. The method according to item 40, wherein the swelling behavior is modified in such a way that:
   the maximum viscosity that is achieved by mixing the synergistic fibre composition, comprising the dietary fibre(s) at an amount x_{_fibre}, in a given volume Vₓ of the aqueous medium (η_{max_syn}) is higher than the maximum viscosity achieved in the same volume Vₓ of the aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}) and higher than η_{max_Σ} (the cumulative maximum viscosity of η_{max_fibre} plus η_{max_other}); and
   the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.
42. The method according to any one of items 40 to 41, wherein the weight ratio of the dietary fibre under (a) to the lipid material under (b) in the synergistic fibre composition is in the range from 30:70 to 60:40, or from 33:67 to 55:45, or from 35:65 to 50:50, or from 35:65 to 45:55, or from 37:63 to 42:58.
43. The method according to any one of items 40 to 42, wherein the synergistic fibre composition comprises at least 45 wt.-%, or at least 50 wt.-%, or at least 55 wt.-%, or at least 60 wt.-% of the lipid material under (b) based on the total weight of the synergistic fibre composition.
44. The method according to any one of items 40 to 43, wherein, the synergistic fibre composition comprises at least 50 wt.-% of the lipid material under (b) based on the total weight of the synergistic fibre composition; and the weight ratio of the dietary fibre under (a) to the lipid material under (b) in the synergistic fibre composition is in the range from 35:65 to 50:50.
45. The method according to any one of items 40 to 44, wherein the dietary fibre under (a) is melt-embedded within, optionally melt-extruded with, the lipid material under (b).
46. The method according to any one of items 40 to 45, wherein the dietary fibre is a so-called viscous fibre, or high-viscosity fibre; for instance, a water-soluble fibre, optionally a water-soluble fibre selected from cellulose ethers, and/or natural gums; optionally uncharged natural gums.
47. The method according to any one of items 40 to 46, wherein the dietary fibre is a water-soluble cellulose ether selected from the group consisting of hydroxpropylmethylcellulose (HPMC), methylcellulose (MC), and carboxymethylcellulose (CMC); and/or
   wherein the dietary fibre is a water-soluble, uncharged natural gum selected from the group consisting of psyllium seed husk, xanthan gum, galactomannan, and glucomannan.
48. The method according to any one of items 40 to 47, wherein the synergistic fibre composition does not comprise any dietary fibres other than a water-soluble cellulose ether selected from the group consisting of hydroxpropylmethylcellulose (HPMC), methylcellulose (MC), and carboxymethylcellulose (CMC); or a water-soluble, uncharged natural gum selected from the group consisting of psyllium seed husk, xanthan gum, galactomannan, and glucomannan.
49. The method according to any one of items 40 to 48, wherein the synergistic fibre composition is substantially free of poly(carboxylates), poly(methacrylic acid), copolymers of acrylic and methacrylic acid, poly(hydroxyethyl methacrylic acid), alginic acid or salts thereof, microcrystalline cellulose, chitosan, gum arabic, beta glucan and pectins.
50. The method according to any one of items 40 to 49, wherein the lipid material under (b) comprises at least one lipid selected from the group consisting of monoglycerides, triglycerides, and sorbitan-fatty acid triesters.
51. The method according to any one of items 40 to 50, wherein the lipid material under (b) comprises at least one lipid selected from the group consisting of monoglycerides, triglycerides, and sorbitan-fatty acid triesters; and wherein the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, of said selected lipid(s), based on the total weight of the lipid material (b).
52. The method according to any one of items 40 to 51, wherein the lipid material under (b₂) is selected from the group consisting of:
   glycerolmonostearate (GMS),
   glycerolmonooleate (GMO),
   tristearin (TS), and
   a triglyceride-based hard fat (Adeps solidus).
53. The method according to any one of items 40 to 52, wherein the lipid material under (b₂) is a triglyceride-based hard fat, and wherein the triglyceride-based hard fat exhibits a melting point in the range of 30 to 45° C. and/or a hydroxylvalue values of 5-50.
54. The method according to any one of items 40 to 53, wherein the lipid material under (b₂) is a triglyceride-based hard fat exhibiting a melting point in the range of 40 to 45° C. and/or a hydroxylvalue values of 5-15.
55. The method according to any one of items 40 to 53, wherein the lipid material under (b₂) is a triglyceride-based hard fat exhibiting a melting point in the range of 32 to 37° C. and/or a hydroxylvalue values of 20-30.
56. The method according to any one of items 40 to 55, wherein the lipid material under (b₂) is selected from the group consisting of:
   glycerolmonostearate (GMS),
   glycerolmonooleate (GMO),
   tristearin (TS), and
   a triglyceride-based hard fat; and
   wherein the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-%, of this/these selected lipid(s), based on the total weight of the lipid material (b).
57. The method according to any one of items 40 to 56, wherein the lipid material under (b) further comprises an additional lipid (b′) in a homogenous mixture with the one or more lipid(s) under b₁, b₂ and/or b₃, wherein additional lipid (b′) is selected from the group consisting of:
   glycerol,
   a phosphatidylcholine,
   a sorbitan mono fatty acid ester,
   one or more fatty acid ester(s) selected from the group consisting of fatty acid esters of acetic acid, lactic acid, citric acid, tartaric acid, monoacetyl tartaric acid and diacetyl tartaric acid,
   one or more fatty acids with a melting point below 30° C.; and
   wherein the homogenous mixture exhibits a melting point in the range of from 30 to 70° C.
58. The method according to any one of items 40 to 57, wherein the lipid material under (b) further comprises an additional lipid (b′) in a homogenous mixture with the one or more lipid(s) under bi, b₂ and/or b₃, wherein the additional lipid (b′) is a phosphatidylcholine, optionally a lecithin such as egg lecithin, soy lecithin, or sunflower lecithin.
59. The method according to any one of items 40 to 58, wherein the lipid material under (b) further comprises an additional lipid (b′) in a homogenous mixture with the one or more lipid(s) under b₁, b₂ and/or b₃, wherein the additional lipid (b′) is a sorbitan mono fatty acid ester, optionally a sorbitan mono fatty acid ester selected from the group consisting of sorbitanmonooleate, polyoxyethylen sorbitanmonolaurate, polyoxyethylen sorbitanmonostearate, and polyoxyethylen sorbitanmonooleate.
60. The method according to any one of items 40 to 59, wherein the lipid material under (b₁) is selected from the group consisting of saturated fatty acids, C₈-C₁₄ fatty acids, and saturated C₈-C₁₄ fatty acids.
61. The method according to any one of items 40 to 60, wherein the lipid material under (b₁) is selected from the group consisting of myristic acid and capric acid.
62. The method according to any one of items 40 to 61, wherein the lipid material under (b₃) is a plant-derived wax selected from the group consisting of candelilla wax, carnauba wax, berry wax, myrica fruit wax, soy wax, rice bran wax, and bees wax.
63. The method according to any one of items 40 to 63, wherein the lipid material under (b₃) is candelilla wax.
64. The method according to any one of items 40 to 63, wherein the lipid material under (b) comprises at least 70 wt.-%, or at least 75 wt.-%, or at least 80 wt.-%, or at least 85 wt.-%, or at least 90 wt.-%, of the lipids (b₁) to (b₃) based on the total weight of the lipid material (b).
65. The method according to any one of items 40 to 64, wherein the lipid material under (b) consists of the lipids (b₁) to (b₃), or
   optionally of the lipids (b₁) to (b₃) and the additional lipids (b′).
66. The method according to any one of items 40 to 65, wherein the lipid material under (b) exhibits a melting point in the range of from 30 to 70° C.
67. The method according to any one of items 40 to 66, wherein the lipid material under (b) comprises at least one lipid selected from the group consisting of myristic acid, capric acid, glycerolmonostearate (GMS), tristearin, and candelilla wax; and wherein optionally the lipid material under (b) comprises at least 50 wt.-%, or at least 55 wt-%, or at least 60 wt.-%, or at least 65 wt.-%, or at least 70 wt.-%, or at least 75 wt.-%, or at least 80 wt-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b).
68. The method according to any one of items 40 to 67, wherein the lipid material under (b) comprises at least one lipid selected from the group consisting of myristic acid, capric acid, glycerolmonostearate (GMS), tristearin, and candelilla wax;
   wherein the lipid material under (b) comprises at least 50 wt.-%, or at least 55 wt.-%, or at least 60 wt.-%, or at least 65 wt-%, or at least 70 wt.-%, or at least 75 wt-%, or at least 80 wt.-%, or at least 85 wt.-%, or at least 90 wt-% of the lipid(s) selected from the group consisting of myristic acid, capric acid, glycerolmonostearate (GMS), tristearin, and candelilla wax, based on the total weight of the lipid material (b); and
   wherein the lipid material under (b) comprises at least one other lipid selected from the group consisting of caprylic acid, glycerol, and glycerolmonooleate (GMO).
69. The method according to any one of items 40 to 68, wherein the lipid material under (b) comprises at least one lipid selected from the group consisting of myristic acid, capric acid, glycerolmonostearate (GMS), tristearin, and candelilla wax; wherein the lipid material under (b) comprises at least 50 wt.-%, or at least 55 wt.-%, or at least 60 wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b);
   wherein the lipid material under (b) comprises at least one other lipid selected from the group consisting of caprylic acid, glycerol, and glycerolmonooleate (GMO); and wherein the lipid material under (b) further comprises lecithin.
70. The method according to any one of items 40 to 69, wherein the synergistic fibre composition further comprises xanthan, either in the form of a powder top-coating layer, and/or with the xanthan being embedded in the lipid(s) under (b) together with the dietary fibre under (a); optionally, in a weight ratio of fibre to xanthan in the range of 98:2 to 75:25, 96:4 to 80:20, or 94:6 to 82:18; such as 91:9 or 85:15.
71. The method according to any one of items 40 to 70, wherein the synergistic fibre composition further comprises xanthan in the form of a powder top-coating layer; and wherein the coating level thereof is in the range of 1 to 20 wt.-%, or 1 to 10 wt.-%, or 2 to 8 wt.-%, based on the total weight of the uncoated cores.
72. The method according to any one of items 40 to 71, wherein the synergistic fibre composition comprises, or consists of, hydroxpropylmethylcellulose (HPMC) as the dietary fibre under (a), and glycerolmonostearate (GMS), glycerolmonooleate (GMO), and lecithin as the components of the lipid material under (b).
73. The method according to any one of items 40 to 72, wherein the synergistic fibre composition comprises, or consists of, hydroxypropylmethylcellulose (HPMC) and xanthan as the dietary fibres under (a); glycerolmonostearate (GMS), glycerolmonooleate (GMO), and lecithin as the components of the lipid material under (b).
74. The method according to any one of items 40 to 73, wherein the synergistic fibre composition comprises, or consists of, hydroxypropylmethylcellulose (HPMC) and xanthan as the dietary fibres under (a); glycerolmonostearate (GMS), glycerolmonooleate (GMO), and lecithin as the components of the lipid material under (b);
   wherein the xanthan is provided either in the form of a powder top-coating layer on the synergistic fibre composition, and/or
   wherein the xanthan is embedded within the lipid material together with the hydroxypropylmethylcellulose (HPMC).
75. The method according to any one of items 40 to 74, wherein the synergistic fibre composition comprises, or consists of, hydroxypropylmethylcellulose (HPMC) and xanthan as the dietary fibres under (a); glycerolmonostearate (GMS), glycerolmonooleate (GMO), and lecithin as the components of the lipid material under (b); and
   wherein the xanthan is provided in the form of a powder top-coating layer on the synergistic fibre composition.
76. The method according to any one of items 40 to 75, wherein, based on its total weight, the synergistic fibre composition comprises, or consists of:
   from 25 to 55 wt.-%, or from 35 to 45 wt.-%, hydroxypropylmethylcellulose (HPMC),
   from 25 to 55 wt.-%, or from 35 to 45 wt.-%, glycerolmonostearate (GMS),
   from 10 to 30 wt-%, or from 15 to 25 wt-%, glycerolmonooleate (GMO),
   from 1 to 20 wt-%, or from 1 to 10 wt.-%, lecithin, and
   from 1 to 20 wt.-%, or from 1 to 10 wt.-%, xanthan.
77. The method according to any one of items 40 to 76, wherein, based on its total weight, the synergistic fibre composition comprises, or consists of:
   from 36 to 40 wt-% hydroxypropylmethylcellulose (HPMC),
   from 36 to 40 wt.-% glycerolmonostearate (GMS),
   from 17 to 21 wt.-%, glycerolmonooleate (GMO),
   from 1 to 3 wt.-%, lecithin, and
   from 1 to 5 wt.-% xanthan.
78. The method according to any one of items 40 to 77, wherein, based on its total weight, the synergistic fibre composition comprises, or consists of:
   from 36 to 40 wt.-% hydroxypropylmethylcellulose (HPMC),
   from 36 to 40 wt.-% glycerolmonostearate (GMS),
   from 17 to 21 wt.-%, glycerolmonooleate (GMO),
   from 1 to 3 wt.-%, lecithin, and
   from 1 to 5 wt.-% xanthan; and the xanthan is provided either in the form of a powder top-coating layer on the synergistic fibre composition and/or embedded in the lipids together with the HPMC is provided in the form of a powder top-coating layer on the synergistic fibre composition.
79. The method according to any one of items 40 to 78, wherein the synergistic fibre composition is provided in the form of a plurality of dry, flowable, ingestible particles, for instance as minitablets, granules, pellets, or powders, said ingestible particles optionally exhibiting a sieve diameter of above 0.01 mm, or above 0.05 mm, or above 0.1 mm, or above 0.2 mm, or above 0.3 mm; and/or a sieve diameter of up to 3.0 mm, or up to 2.0 mm, or up to 1.5 mm, or up to 1.0 mm, or up to 0.8 mm, or up to 0.6 mm, or up to 0.5 mm,
   such as a sieve diameter in the range of 0.01 mm-3.0 mm, or 0.1 mm-3.0 mm, or 0.5 mm-3.0 mm, or 0.75 mm-2.5 mm, or 0.1 mm-2.0 mm, or 0.5 mm-2.0 mm, or 0.1 mm-2.0 mm, or 0.1 mm-1.5 mm, or 0.1 mm-1.0 mm, or 0.1 mm-0.8 mm, or 0.2 mm-1.0 mm, or 0.2 mm-0.8 mm, 0.5 mm-2 mm, or 0.5 mm-1.5 mm, or 0.5 mm-1.25 mm, or 0.5 mm-1.0 mm.
80. The method according to any one of items 40 to 79, wherein the synergistic fibre composition is provided in the form of a plurality of dry, flowable, ingestible particles, for instance as minitablets, granules, pellets, or powders, and said ingestible particles are free of a synthetic drug substance.
81. The method according to any one of items 40 to 80, wherein the viscosity in the aqueous medium is measured at a temperature in the range of from 15 to 40° C.
82. The method according to any one of items 40 to 81, wherein the viscosity in the aqeuous medium is measured at room temperature, or at a temperature of about 37° C.
83. The method according to any one of items 40 to 82, wherein the viscosity in the aqeuous medium is measured at room temperature, or at a temperature of about 37° C., and wherein the aqueous medium is selected from water, fasted-state simulated intestinal fluid (FaSSIF), fed-state simulated intestinal fluid (FeSSIF), fasted-state simulated intestinal fluid (FaSSGF), 0.1 N HCl, pH 6.8 phosphate-buffer solution, or similar bio-relevant media.
84. The method according to any one of items 40 to 83, wherein, in water at room temperature, η_{max_syn} (the maximum viscosity that is achieved by mixing the synergistic fibre composition, comprising the dietary fibre(s) at an amount x_{_fibre}, in a given volume Vₓ of the water) is at least 10%, preferably at least 15%, more preferably at least 20% higher, than η_{max_Σ} (the cumulative maximum viscosity of η_{max_fibre} plus η_{max_other}).
85. The method according to any one of items 40 to 84, wherein the synergistic fibre composition further comprises xanthan, and
   wherein, in water at room temperature, η_{max_syn} is at least 30%, preferably at least 40%, more preferably at least 50%, higher than η_{max_Σ}.
86. The method according to any one of items 40 to 85, wherein the synergistic fibre composition further comprises xanthan in the form of a powder top-coating layer, at a coating level in the range of 1 to 20 wt.-%, or 1 to 10 wt.-%, or 2 to 8 wt.-%, based on the total weight of the uncoated cores, and
   wherein, in water at room temperature, η_{max_syn} is at least 30%, preferably at least 40%, more preferably at least 50%, higher than η_{max_Σ} (the cumulative maximum viscosity of η_{max_fibre} plus η_{max_other}).
87. The method according to any one of items 40 to 86, wherein the swelling exhibits sigmoidal viscosity kinetics characterized by a lag time of at least 20 min, followed by a viscosity increase to at least 90% of the maximum viscosity (η_{max_syn}), that is achieved by mixing the synergistic fibre composition, comprising the dietary fibre(s) at an amount x_{_fibre}, in a given volume Vₓ of the aqueous medium, within no more than 2 h.
88. The method according to any one of items 40 to 87, wherein the swelling behavior is modified in such a way that in water at room temperature:
   the maximum viscosity that is achieved by mixing the synergistic fibre composition, comprising the dietary fibre(s) at an amount x_{_fibre}, in a given volume Vₓ of the water (η_{max_}syn) is at least 10%, preferably at least 15%, more preferably at least 20% higher than the cumulative maximum viscosity of η_{max_fibre} plus η_{max_other} (η_{max_Σ}); and
   the swelling exhibits sigmoidal viscosity kinetics characterized by a lag time of at least 20 min, followed by a viscosity increase to at least 90% of the maximum viscosity (η_{max_syn}), that is achieved by mixing the synergistic fibre composition, comprising the dietary fibre(s) at an amount x_{_fibre}, in a given volume V. of the water, within no more than 2 h.
89. A process for preparing the synergistic fibre composition provided for the method according to items 40 to 88, or specifically the synergistic fibre composition according to items 1 to 39, wherein the process comprises a step of processing a blend comprising the dietary fibre under (a) and the lipid material under (b) by:
   (i) extruding, or melt-extruding, the blend;
   (ii) spray-congealing the blend, optionally using a jet-break-up technique;
   (iii) melt-granulating the blend;
   (iv) compressing the blend into minitablets;
   (v) melt-injecting the blend into a liquid medium; or
   (vi) spray-coating the blend onto inert cores.
90. The process according to item 89, wherein the extrusion, or melt-extrusion, under (i) is carried out in twin-screw extruder.
91. The composition according to any one of items 1 to 39 for use as a medicament.
92. The composition according to any one of items 1 to 39 for use in the treatment and/or prevention of gastro-intestinal and/or metabolic disorders.
93. The composition for use according to any item 92, wherein of gastro-intestinal and/or metabolic disorder is selected from the group consisting of constipation, diverticulosis, irritable bowel syndrome and Crohn's disease, elevated plasma cholesterol levels, metabolic syndrome, pre-diabetes, diabetes, specifically diabetes type 2, overweight, and obesity.
94. A solid dietary fibre formulation for oral administration comprising the synergistic fibre composition provided for the method according to items 40 to 88, or specifically the synergistic fibre composition according to items 1 to 39, wherein the synergistic fibre composition is provided in the form of a plurality of dry, flowable, ingestible particles, for instance as minitablets, granules, pellets, or powders, or has been prepared from said plurality of dry, flowable, ingestible particles.

1. A synergistic fibre composition comprising:
(a) a dietary fibre selected from the group consisting of hydroxpropylmethylcellulose (HPMC), methylcellulose (MC), carboxymethylcellulose (CMC), psyllium seed husk, xanthan gum, galactomannan, and glucomannan; and
(b) a lipid material comprising a lipid selected from:
(b₁) a free fatty acid exhibiting a melting point in the range of from 30 to 60° C.,
(b₂) a glycerol- or sorbitan based fatty acid ester exhibiting a melting point in the range of from 30 to 75° C.,
(b₃) a plant-derived wax exhibiting a melting point in the range of from 30 to 75° C., or
a homogenous mixture of two or more of the lipids under b₁, b₂ and/or b₃, wherein the homogenous mixture of these lipids exhibit a melting point in the range of from 30 to 70° C.,
wherein the lipid material under (b) comprises at least one lipid under b₁ to b₃ which is selected from the group consisting of myristic acid, capric acid, glycerolmonostearate (GMS), tristearin, and candelilla wax;
wherein the dietary fibre is embedded within the lipid material; and
wherein the synergistic fibre composition is capable of modifying the swelling behavior of the dietary fibre in such a way that:
the maximum viscosity that is achieved by mixing the synergistic fibre composition, comprising the dietary fibre(s) at an amount x_{_fibre}, in a given volume Vₓ of the aqueous medium (η_{max_syn}) is higher than the maximum viscosity achieved in the same volume Vₓ of an aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}) and higher than the cumulative maximum viscosity of η_{max_fibre} plus η_{max_other} (η_{max_Σ}); and optionally
the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.

2. The composition according to claim 1, wherein the weight ratio of the dietary fibre under (a) to the lipid material under (b) in the synergistic fibre composition is in the range from 30:70 to 60:40, or from 33:67 to 55:45, or from 35:65 to 50:50, or from 35:65 to 45:55, or from 37:63 to 42:58.

3. The composition according to claim 1, wherein the synergistic fibre composition comprises at least 45 wt.-%, or at least 50 wt.-%, or at least 55 wt.-%, or at least 60 wt.-% of the lipid material under (b) based on the total weight of the synergistic fibre composition.

4. The composition according to claim 1, wherein the dietary fibre under (a) is melt-embedded within, optionally melt-extruded with, the lipid material under (b).

5. The composition according to claim 1, wherein the synergistic fibre composition does not comprise any dietary fibres other than a dietary fibre selected from the group consisting of hydroxypropylmethylcellulose (HPMC), methylcellulose (MC), carboxymethylcellulose (CMC), psyllium seed husk, xanthan gum, galactomannan, and glucomannan.

6. The composition according to claim 1, wherein the lipid material under (b) comprises at least 50 wt.-%, or at least 55 wt.-%, or at least wt.-%, or at least 65 wt.-%, or at least 70 wt.-%, or at least 75 wt.-%, or at least 80 wt.-%, or at least 85 wt.-%, or at least 90 wt.-% of the lipid(s) selected from the group consisting of myristic acid, capric acid, glycerolmonostearate (GMS), tristearin, and candelilla wax, based on the total weight of the lipid material (b).

7. The composition according to claim 1, wherein the lipid material under (b) further comprises an additional lipid (b′) in a homogenous mixture with the one or more lipid(s) under b₁, b₂ and/or b₃, wherein additional lipid (b′) is selected from the group consisting of:
glycerol,
a phosphatidylcholine,
a sorbitan mono fatty acid ester,
one or more fatty acid ester(s) selected from the group consisting of fatty acid esters of acetic acid, lactic acid, citric acid, tartaric acid, monoacetyl tartaric acid and diacetyl tartaric acid,
one or more fatty acids with a melting point below 30° C.; and
wherein the homogenous mixture exhibits a melting point in the range of from 30 to 70° C.

8. The composition according to claim 1, wherein the lipid material under (b) comprises at least 70 wt.-%, or at least 75 wt.-%, or at least 80 wt.-%, or at least 85 wt.-%, or at least 90 wt.-%, of the lipids (bi) to (b₃) based on the total weight of the lipid material (b).

9. The composition according to claim 1, wherein the lipid material under (b) consists of the lipids (b₁) to (b₃), or optionally of the lipids (b₁) to (b₃) and the additional lipids (b′).

10. The composition according to claim 1, wherein the synergistic fibre composition further comprises xanthan, either in the form of a powder top-coating layer, and/or with the xanthan being embedded in the lipid(s) under (b) together with the dietary fibre under (a); optionally, in a weight ratio of fibre to xanthan in the range of 98:2 to 75:25.

11. The composition according to claim 1, wherein the synergistic fibre composition comprises, or consists of, hydroxpropylmethylcellulose (HPMC), and optionally xanthan, as the dietary fibre(s) under (a); and glycerolmonostearate (GMS), glycerolmonooleate (GMO), and lecithin as the components of the lipid material under (b).

12. The composition according to claim 1, wherein the synergistic fibre composition is provided in the form of a plurality of dry, flowable, ingestible particles, such as minitablets, granules, pellets, or powders; and wherein optionally said ingestible particle are free of a synthetic drug substance.

13. A method of modifying the swelling behavior of a dietary fibre in an aqueous medium by providing a synergistic fibre composition comprising:
(a) the dietary fibre, and
(b) a lipid material comprising a lipid selected from:
(b₁) a free fatty acid exhibiting a melting point in the range of from 30 to 60° C.,
(b₂) a glycerol- or sorbitan based fatty acid ester exhibiting a melting point in the range of from 30 to 75° C.,
(b₃) a plant-derived wax exhibiting a melting point in the range of from to 75° C., or
a homogenous mixture of two or more of the lipids under b₁, b₂ and/or b₃, wherein the homogenous mixture of these lipids exhibits a melting point in the range of from 30 to 70° C.;
wherein the dietary fibre is embedded within the lipid material, and
wherein the swelling behavior is modified in such a way that:
the maximum viscosity that is achieved by mixing the synergistic fibre composition, comprising the dietary fibre(s) at an amount x_{_fibre}, in a given volume Vₓ of the aqueous medium (η_{max_syn}) is higher than the maximum viscosity achieved in the same volume Vₓ of the aqueous medium by the dietary fibre(s) alone at the same amount x_{_fibre} (η_{max_fibre}), higher than the maximum viscosity achieved in the same volume Vₓ of the aqueous medium by any of the other components of the synergistic fibre composition (η_{max_other}), and higher than the cumulative maximum viscosity of η_{max_fibre} plus η_{max_other} (η_{max_Σ}); and optionally
the swelling exhibits sigmoidal viscosity kinetics characterized by a lag time of at least 15 min, followed by a viscosity increase to at least 80% of the maximum viscosity (η_{max_syn}), that is achieved by mixing the synergistic fibre composition comprising the dietary fibre(s) at an amount x_{_fibre} in a given volume Vₓ of the aqueous medium, within no more than 2 h.

14. (canceled)

15. A method of treatment and/or prevention of gastro-intestinal and/or metabolic disorders, the method comprising administering a therapeutically effective amount of the composition according to claim 1 to a subject in need thereof.

16. A solid dietary fibre formulation for oral administration comprising, or consisting of, the synergistic fibre composition according to claim 1, wherein the synergistic fibre composition is provided in the form of a plurality of dry, flowable, ingestible particles, for instance as minitablets, granules, pellets, or powders, or has been prepared from said plurality of dry, flowable, ingestible particles.

17. A method of treatment and/or prevention of gastro-intestinal and/or metabolic disorders, the method comprising administering a therapeutically effective amount of the synergistic fibre composition provided for by the method of claim 13 to a subject in need thereof.

18. A solid dietary fibre formulation for oral administration comprising, or consisting of, the synergistic fibre composition provided for by the method of claim 13, wherein the synergistic fibre composition is provided in the form of a plurality of dry, flowable, ingestible particles, for instance as minitablets, granules, pellets, or powders, or has been prepared from said plurality of dry, flowable, ingestible particles.
